(19) United States
(12) Patent Application Publication
(10) Pub. No.: US 2008/0253985 A1
(43) Pub. Date: Oct. 16, 2008

(54) Compositions for Lowering Serum Cholesterol and/or Triglycerides

(76) Inventor: Gerald L. Wisler, Gladstone, NJ (US)

Correspondence Address: GOODWIN PROCTER LLP;PATENT ADMINISTRATOR, EXCHANGE PLACE, BOSTON, MA, 02109-2881, US

(21) Appl. No.: 12/090,539

(22) PCT Filed: Oct. 18, 2006
(86) PCT No.: PCT/US06/40953

§ 371 Date: Jun. 27, 2008

Related U.S. Application Data
(60) Provisional application No. 60/727,664, filed on Oct. 18, 2005.
(60) Provisional application No. 60/788,616, filed on Apr. 3, 2006.

(51) Int. Cl. A61K 31/785; A61K 31/4468; A61K 31/455; A61K 31/437; A61K 31/192; A61P 9/10; A61K 31/216; A61K 31/351; A61K 31/40; A61K 31/397

(52) U.S. Cl. 424/78.16; 514/325; 514/356; 514/292; 514/571; 514/460; 514/423; 514/210.02; 514/545

(57) ABSTRACT

The invention provides methods and compositions for treating hyperlipidemia and disorders associated with hyperlipidemia in a mammal. Compositions useful in the practice of the invention include a microsomal triglyceride transport protein inhibitor (“MTPI”) and at least two other cholesterol lowering drugs selected from the group consisting of a cholesterol absorption inhibitor (“CAI”), a HMG-CoA reductase inhibitor, a bile acid sequestrant, a fibric acid derivative, niacin, and squalene synthetase inhibitor.

### RELATED APPLICATIONS

This application claims the benefit of U.S. Provisional Patent Application Ser. No. 60/788,616, filed Apr. 3, 2006, and U.S. Provisional Patent Application Ser. No. 60/727,664, filed Oct. 18, 2005, the entire disclosures of which are incorporated by reference herein.

### FIELD OF THE INVENTION

The present invention relates generally to the field of pharmaceutical compositions and their use in the treatment of hyperlipidemia, and more particularly relates to therapeutic combinations comprising a microsomal triglyceride transfer protein inhibitor and at least two other cholesterol lowering agents, and their use in the treatment of hyperlipidemia.

### BACKGROUND OF THE INVENTION

There are several known risk factors for atherosclerotic cardiovascular disease (ASCVD), the major cause of mortality in the Western world. One key risk factor is hyperlipidemia, which is the presence of elevated levels of lipids in blood plasma. Various epidemiological studies have demonstrated that drug mediated lowering of total cholesterol (TC) and low density lipoprotein (LDL) cholesterol (LDL-C) is associated with a significant reduction in cardiovascular events. The National Cholesterol Education Program's (NCEP's) updated guidelines recommends that the overall goal for high-risk patients is to achieve less than 100 mg/dL of LDL, with a therapeutic option to set the goal for such patients to achieve a LDL level less than 70 mg/dL.

One form of hyperlipidemia is known as hypertriglyceridemia and results in the presence of elevated amounts of triglycerides in the blood. Although triglycerides are necessary for good health, higher-than-normal triglyceride levels, often are associated with known risk factors for heart disease.

Another form of hyperlipidemia, known as hypercholesterolemia, which is the presence of elevated amounts of cholesterol in the blood, is a polygenic disorder. Modifications in lifestyle and conventional drug treatment are usually successful in reducing cholesterol levels. However, in some cases, as in familial hypercholesterolemia (FH), the cause is a monogenic defect. Treatment of a patient with FH can be more challenging because the levels of LDL-C remain elevated despite aggressive use of conventional therapy.

For example, one type of FH, homozygous familial hypercholesterolemia (hoFH), is a serious life-threatening genetic disease caused by homozygosity or compound heterozygosity for mutations in the low density lipoprotein (LDL) receptor. Patients with hoFH typically have total plasma cholesterol levels over 400 mg/dL resulting in premature atherosclerotic vascular disease. When left untreated, most patients develop atherosclerosis before age 20 and generally do not survive past age 30. However, patients diagnosed with hoFH are largely unresponsive to conventional drug therapy and have limited treatment options. Specifically, treatment with statins, which reduce LDL-C by inhibiting cholesterol synthesis and upregulating the hepatic LDL receptor, have negligible effect in patients whose LDL receptors are non-existent or defective. A mean LDL-C reduction of only less than about 20% has been recently reported in patients with genotype-confirmed hoFH treated with the maximal dose of statins (atorvastatin or simvastatin administered at 80 mg/day). The addition of ezetimibe 10 mg/day to this regimen resulted in a total reduction of LDL-C levels of 27%, which is still far from optimal. Non-pharmacological options have also been tested, including surgical interventions, such as portacaval shunt and ileal bypass, and orthotopic liver transplantation, but with clear disadvantages and risks. Therefore, there is a tremendous unmet medical need for new medical therapies for hoFH.

Microsomal triglyceride transfer protein (MTP) inhibitors have been developed as potent inhibitors of MTP-mediated neutral lipid transfer activity. MTP catalyzes the transport of triglyceride, cholesteryl ester, and phosphatidylcholine between small unilamellar vesicles. One exemplary MTP inhibitor is BMS-201038, developed by Bristol-Myers Squibb. See, U.S. Pat. Nos. 5,739,135; and 5,712,279. Studies using an animal model for homozygous FH indicated that BMS-201038 effectively reduced plasma cholesterol levels in a dose dependent manner, for example, at 25 mg/day, suggesting that this compound might be effective for treating patients with hoFH. It was noticed, however, that certain patients treated with 25 mg/day of BMS-201038 experienced certain adverse events, for example, gastrointestinal disturbances, abnormalities in liver function, and hepatic steatosis. Although a promising therapeutic agent, large scale clinical trials of BMS-201038 have been discontinued. Another potent MIP inhibitor known as implitapide has been developed. See, U.S. Pat. Nos. 6,265,431, 6,479,503, 5,952,498. During clinical studies, dosages of implitapide of 80 mg/day or greater, although therapeutically effective, were found to result in certain adverse events, for example, gastrointestinal disturbances, abnormalities in liver function, and hepatic steatosis. Large scale clinical studies using implitapide have also been discontinued.

Accordingly, there is still a need for methods for aggressively treating hyperlipidemias that effectively lower, for example, circulating cholesterol and triglycerides levels so as to improve the rates of achieving goals of therapy based on published guidelines, for example, NCEP guidelines, but with fewer or reduced adverse effects that typically result when higher dosages of the MTP inhibitor are used alone in monotherapy.

### BRIEF SUMMARY OF THE INVENTION

The invention is based, in part, upon the development of compositions comprising an MTP inhibitor in combination with at least two other cholesterol lowering agents. It is contemplated that the combination of active ingredients will not only provide a greater degree of goal attainment, but it will also permit the goals to be achieved at lower dosages of the individual active ingredients thereby reducing the incidence and/or severity of dose-related adverse events associated with the individual active ingredients. It is contemplated that, for example, lowering blood LDL levels below those already achieved in earlier clinical trials by using, for example, an MTP inhibitor in combination with a HMG-CoA reductase inhibitor plus a cholesterol absorption inhibitor (CAI) will provide further improvements in cardiovascular event rate reduction and/or plaque regression.

For example, the compositions can be used to reduce the fasting levels of cholesterol and/or triglycerides in the blood of a mammal to meet a clinical endpoint but with fewer or reduced adverse events than (i) when the MTP inhibitor is administered alone in a monotherapy at a dosage sufficient to meet the clinical endpoint or (ii) when the MTP inhibitor is administered together with another cholesterol lowering agent, where the MTP inhibitor and the other cholesterol lowering agent are administered at dosages sufficient to meet the clinical end point.

Furthermore, the compositions can be used to reduce by at least 55%, 60%, or 65%, the blood LDL concentration in a population of patients who, prior to therapy have circulating LDL concentrations of at least 130 mg/dL, so as to meet the goal of having an LDL concentration of 70 mg/dL or less, where (i) less than 2% of the patients in the population have Liver Function Test results three times greater than the upper limit of normal of a standard clinical laboratory range or (ii) the patients have statistically significant lower rates of skeletal muscle side effects (e.g., myalgia and/or myopathy) relative to patients receiving the maximum permitted dose of a HMG-CoA reductase inhibitor. In this context, the term “permitted” refers to a maximum dosage permitted by a regulatory agency, for example, the U.S. Food and Drug Agency.

Furthermore, it is contemplated that the compositions, when administered to the recipient, will not only permit the recipient to meet a cholesterol goal but will also slow down or stop the build up of plaques, for example, atherosclerotic plaques, on the walls of blood vessels. Under certain circumstances, it is contemplated that the compositions, when administered, will also induce regression of existing plaques.

In one aspect, the invention provides a pharmaceutical composition comprising (i) a MTP inhibitor, (ii) a CAI, and (iii) at least one cholesterol lowering drug selected from the group consisting of a HMG-CoA reductase inhibitor, a bile acid sequestrant, a fibric acid derivative, niacin, and a squalene sythetase inhibitor. In another aspect, the invention provides a pharmaceutical composition comprising (i) an MTPI, (ii) a HMG-CoA reductase inhibitor, and (iii) at least one cholesterol lowering drug selected from the group consisting of a bile acid sequestrant, a fibric acid derivative, niacin, and a squalene sythetase inhibitor.

It is possible that the pharmaceutical composition can comprise an MTPI, (ii) a CAI, (iii) a HMG-CoA reductase inhibitor, and (iv) a cholesterol lowering drug selected from the group consisting of a bile acid sequestrant, a fibric acid derivative, and niacin.

The MTPI can be selected from known compounds selected from the group consisting of BMS-201038, implitapide, JTT-130 and CP-346086, and SLx-4090. The HMG-CoA reductase inhibitor can be selected from the group consisting of mevastatin, lovastatin, pravastatin, simvastatin, fluvastatin, cerivastatin, atorvastatin, tenivastatin, rosuvastatin, pitavastatin. The CAI can be selected from the group consisting of ezetimibe or a derivative thereof, MD-0727, FM-VP4, LPD-179, LPD84, and LPD145. The bile acid sequestrant can be selected from the group consisting of cholestyramine, colesevelam and colestipol. The fibric acid derivative can be selected from the group consisting of fenofibrate, bezafibrate, ciprofibrate, clofibrate, and gemfibrozil.

### DETAILED DESCRIPTION OF THE INVENTION

### (1) Definitions

For convenience, certain terms used in the specification, examples, and appended claims are collected here.

The phrase “combination therapy,” as used herein, refers to co-administering an MTP inhibitor and at least two other cholesterol lowering agents, for example, where one is a HMG Co-A reductase and the other is a CAI, as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually weeks, months or years depending upon the combination selected). Combination therapy is intended to embrace administration of multiple therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single tablet or capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection.

Combination therapy also can embrace the administration of the therapeutic agents as described above in further combination with other biologically active ingredients and non-drug therapies. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

The components of the combination may be administered to a patient simultaneously or sequentially. It will be appreciated that the components may be present in the same pharmaceutically acceptable carrier and, therefore, are administered simultaneously. Alternatively, the active ingredients may be present in separate pharmaceutical carriers, such as, conventional oral dosage forms, that can be administered either simultaneously or sequentially.

The terms, “individual,” “patient,” or “subject” are used interchangeably herein and include any mammal, including animals, for example, primates, for example, humans, and other animals, for example, dogs, cats, swine, cattle, sheep, and horses. The compounds of the invention can be administered to a mammal, such as a human, but can also be other mammals, for example, an animal in need of veterinary treatment, for example, domestic animals (for example, dogs, cats, and the like), farm animals (for example, cows, sheep, pigs, horses, and the like) and laboratory animals (for example, rats, mice, guinea pigs, and the like).

The term, “patient resistant to statin monotherapy,” as used herein includes those patients for whom conventional statin monotherapy has been found ineffective or less effective than desired. A physician designing lipid reduction therapy for a patient will be able to determine via diagnosis and observation of periodic blood cholesterol and/or triglyceride levels whether such a patient is or has been resistant to statin monotherapy.

The term, “statin-intolerant patient,” as used herein includes those patients for whom conventional statin therapy, for example, for serum lipid reduction, has been found to be ineffective and/or for whom an effective lipid-reducing dose of statins is too high to be tolerated or that there is an unacceptable adverse event associated with a particular dose. For example, statin therapy may be discontinued by the physician/patient due to concern over an adverse event such as Liver Function Test abnormality, muscle aches and pains or inflammation—myalgia or myostitis, elevation in enzymes (CK) showing muscle adverse event. A physician designing lipid reduction therapy for a patient will be able to determine via diagnosis and observation of periodic blood cholesterol and/or triglyceride levels whether such a patient is statin-intolerant.

The phrase “minimizing adverse effects,” “reducing adverse events,” or “reduced adverse events,” as used herein refer to an amelioration or elimination of one or more undesired side effects associated with the use of MTP inhibitors of the present invention. Side effects of traditional use of the MTP inhibitors include, without limitation, nausea, gastrointestinal disorders, steatorrhea, abdominal cramping, distention, elevated liver function tests, fatty liver (hepatic steatosis); hepatic fat build up, polyneuropathy, peripheral neuropathy, rhabdomyolysis, arthralgia, myalgia, chest pain, rhinitis, dizziness, arthritis, peripheral edema, gastroenteritis, liver function tests abnormal, colitis, rectal hemorrhage, esophagitis, eructation, stomatitis, biliary pain, cheilitis, duodenal ulcer, dysphagia, enteritis, melena, gum hemorrhage, stomach ulcer, tenesmus, ulcerative stomatitis, hepatitis, pancreatitis, cholestatic jaundice, paresthesia, amnesia, libido decreased, emotional lability, incoordination, torticollis, facial paralysis, hyperkinesia, depression, hypesthesia, hypertonia, leg cramps, bursitis, tenosynovitis, myasthenia, tendinous contracture, myositis, hyperglycemia, creatine phosphokinase increased, gout, weight gain, hypoglycemia, anaphylaxis, angioneurotic edema, and bullous rashes (including erythema multiforme, Stevens-Johnson syndrome, and toxic epidermal necrolysis). Accordingly, the methods described herein provide an effective therapy while at the same time causing fewer or less significant adverse events.

In certain embodiments, side effects are partially eliminated. As used herein, the phrase “partially eliminated” refers to a reduction in the severity, extent, or duration of the particular side effect by at least 30%, 40%, 50%, 60%, 70%, 80%, 90%, 95% and 99% relative to that found by administering 25 mg/day of BMS-201038 during monotherapy or either 80 mg/day or 160 mg/day of implitapide during monotherapy. In certain embodiments, side effects are completely eliminated. Those skilled in the art are credited with the ability to detect and grade the severity, extent, or duration of side effects as well as the degree of amelioration of a side effect. In some embodiments, two or more side effects are ameliorated.

The term, “therapeutically effective” refers to the ability of an active ingredient, for example, BMS-201038 and implitapide, to elicit the biological or medical response that is being sought by a researcher, veterinarian, medical doctor or other clinician. Non-limiting examples include reduction of cholesterol (for example, LDL-C) and/or triglyceride levels in a patient, reduction of the amount of plaques, for example, arterial plaques, on the wall of a blood vessel, and the like.

The term, “therapeutically effective amount” includes the amount of an active ingredient, for example, BMS-201038 and implitapide, that will elicit the biological or medical response that is being sought by the researcher, veterinarian, medical doctor or other clinician. The compounds of the invention are administered in amounts effective at lowering the cholesterol concentration in the blood, and/or the triglyceride concentration in the blood and/or reducing the amount of plaques, for example, arterial plaques disposed upon the blood contacting wall of one or more blood vessels. Alternatively, a therapeutically effective amount of an active ingredient is the quantity of the compound required to achieve a desired therapeutic and/or prophylactic effect, such as the amount of the active ingredient that results in the prevention of or a decrease in the symptoms associated with the condition (for example, to meet an end-point).

The terms, “pharmaceutically acceptable” or “pharmacologically acceptable” refer to molecular entities and compositions that do not produce an adverse, allergic or other untoward reaction when administered to an animal, or to a human, as appropriate. The term, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The terms “treating” or “treatment”, refers to any effect, for example, lessening, inhibiting, reducing, modulating, or eliminating, that results in the improvement of the condition, disease, or disorder.

### (2) Formulations for Combination Therapy

The compositions provided herein are useful for treating a number of disorders associated with elevated levels of cholesterol and/or triglycerides in the blood. The compositions comprise an MTP inhibitor in combination with at least two other cholesterol lowering drugs.

In one aspect, the composition comprises (i) an MTP inhibitor, (ii) a CAI, and (iii) at least one cholesterol lowering drug selected from the group consisting of a HMG-CoA reductase inhibitor, a bile acid sequestrant, a fibric acid derivative, niacin, and squalene sythetase inhibitor. When three active ingredients are used, this is referred to as a triple combination. However, it is contemplated that more than three of the active ingredients can be used in the practice of the invention.

In another aspect, the composition comprises (i) and MTP inhibitor, (ii) a HMG-CoA reductase inhibitor, and (iii) at least one cholesterol lowering drug selected from the group consisting of a bile acid sequestrant, a fibric acid derivative, niacin, and squalene synthetase inhibitor. It is contemplated that more than three of the active ingredients can be used in the practice of the invention.

It is contemplated that the combination of active ingredients will not only provide a greater degree of goal attainment, but it will also permit the goals to be achieved at lower dosages of the individual active ingredients thereby reducing the incidence and/or severity of dose-related adverse events associated with the individual active ingredients. It is contemplated that, for example, lowering blood LDL levels below those already achieved in earlier clinical trials by using, for example, an MTP inhibitor in combination with a HMG-CoA reductase inhibitor plus a CAI will provide further improvements in cardiovascular event rate reduction and/or plaque regression

For example, the compositions can be used to reduce the fasting levels of cholesterol and/or triglycerides in the blood of a mammal to meet a clinical end-point but with fewer or reduced adverse events than (i) when the MTP inhibitor is administered alone in a monotherapy at a dosage sufficient to achieve or substantially achieve (for example, within 10%) the clinical end-point or (ii) when the MTP inhibitor is administered together with another cholesterol lowering agent, where the MTP inhibitor and the other cholesterol lowering agent are administered at dosages sufficient to achieve or substantially achieve the clinical end-point.

Furthermore, the compositions can be used to reduce by at least 55%, 60%, or 65%, the blood LDL concentration in a population of patients who, prior to therapy have circulating LDL concentrations of at least 130 mg/dL, so as to meet the goal of having an LDL concentration of 70 mg/dL or less, where (i) less than 2% of the patients in the population have Liver Function Test results three times greater than the upper limit of normal of a standard clinical laboratory range or (ii) the patients have statistically significant lower rates of skeletal muscle side effects (e.g., myalgia and/or myopathy) relative to patients receiving the maximum permitted dose of a HMG-CoA reductase inhibitor.

Furthermore, it is contemplated that the compositions, when administered to the recipient, will not only permit the recipient to meet a cholesterol goal but will also slow down or stop the build up of plaques, for example, atherosclerotic plaques, on the walls of blood vessels. Under certain circumstances, it is contemplated that the compositions, when administered, will also induce regression of existing plaques.

Preferred active agents that can be combined to meet the clinical end points described herein are set forth below.

A. MTP Inhibitors

In one embodiment, the MTP inhibitor may be BMS 201038 (denoted as M1). As used herein, the phrase “BMS-201038” refers to a compound known as N-(2,2,2-Trifluorethyl)-9-[4-[4-[[[4′-(trifluoromethyl)[1,1′biphenyl]-2-Yl]carbonyl]amino]-1-piperidinyl]butyl]9H-fluorene-9-carboxamide, having the formula:

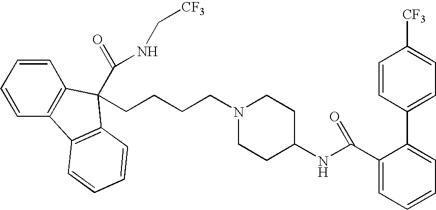

and stereoisomers thereof, and pharmaceutically acceptable salts and esters thereof.

In another embodiment, the MTP inhibitor may include benzimidazole-based analogues of BMS 201038 (denoted as M2). As used herein, the “M2” refers to a compound having the formula shown below:

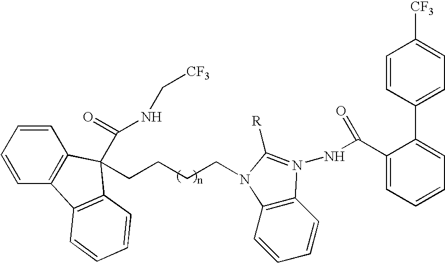

where n can be 0 to 10, and stereoisomers thereof, and pharmaceutically acceptable salts and esters thereof.

In another embodiment, the MTP inhibitor may be implitapide (denoted as M3). As used herein, the phrase “implitapide” refers to a compound (2S)-2-cyclopentyl-2-[4-[(2,4-dimethyl-9H-pyrido[2,3-b]indol-9-yl)methyl]phenyl]-N-[(1S)-2-hydroxy-1-phenylethyl]ethanamide, and having the structure shown below:

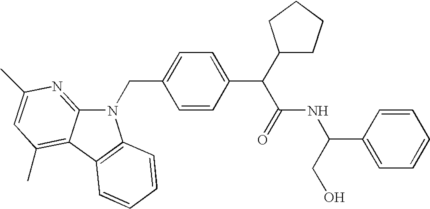

and stereoisomers thereof, and pharmaceutically acceptable salts and esters thereof.

In another embodiment, the MTP inhibitor may be JTT-130m (denoted as M4) including pharmaceutically acceptable salts and esters thereof, described in Aggarwal, et al., BMC CARDIOVASC. DISORD. 27;5(1):30 (2005). In another embodiment, the MTP inhibitor may be CP-346086 (denoted M5) including pharmaceutically salts and esters thereof, described in Chandler, et al., J. LIPID. RES. 44(10):1887-901 (2003).

Other MTP inhibitors include those developed by Surface Logix, Inc. e.g., SLx-4090 (denoted as M6).

B. Other Cholesterol Lowering Agents

Cholesterol lowering agents that may be used in the compositions and methods described herein include:

1. Cholesterol Absorption Inhibitors (CAI)

In one embodiment, the CAI may be ezetimibe (also known as Zetia) (denoted as C1), As used herein, the phrase “ezetimibe” refers to a compound having the structure shown below:

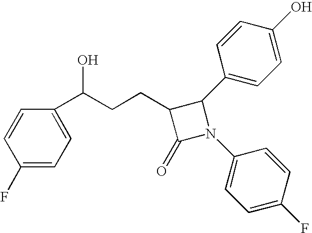

and stereoisomers thereof, and pharmaceutically acceptable salts and esters thereof.

In one embodiment, the CAI may be MD-0727 (denoted as C2) including pharmaceutically acceptable salts and esters thereof. In another embodiment, the CAI may be FM-VP4 (denoted as C3). As used herein, the phrase “FM-VP4” refers to a compound the structure of which is set forth below:

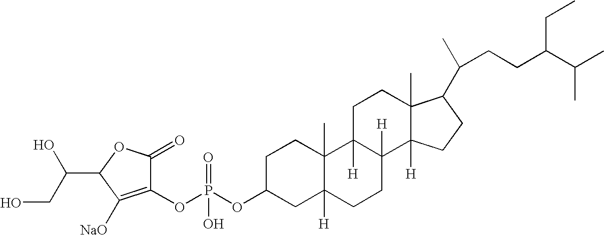

and stereoisomers thereof, and pharmaceutically acceptable salts and esters thereof.

In another embodiment, the CAI may be the structure below (denoted as C4), as described in Ritter et al., Org. Biomol. Chem., 3(19), 3514-3523, (2005):

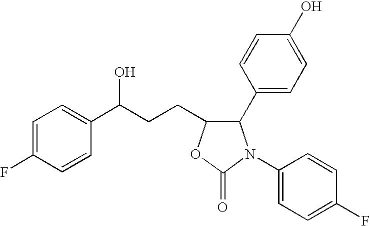

and stereoisomers thereof, and pharmaceutically acceptable salts and esters thereof.

In another embodiment, the CAI may be LPD179 (denoted as C5). As used herein, the phrase “LDP179” refers to a compound having the structure set forth below:

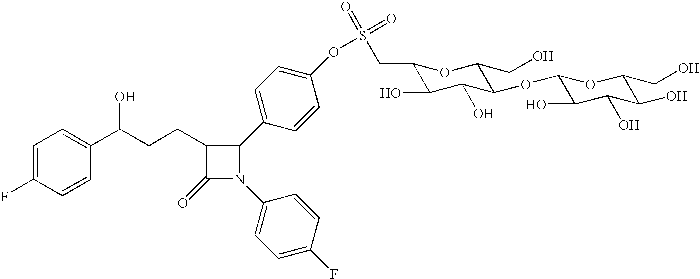

and stereoisomers thereof, and pharmaceutically acceptable salts and esters thereof.

In another embodiment, the CAI may be LPD84 (denoted as C6). As used herein, the phrase “LPD84” refers to a compound having the structure set forth below:

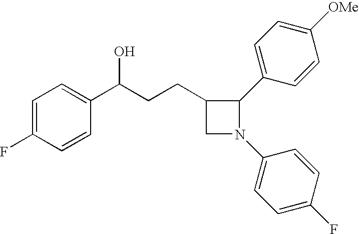

and stereoisomers thereof, and pharmaceutically acceptable salts and esters thereof.

In another embodiment, the CAI may be LPD145 (denoted as C7). As used herein, the phrase “LPD145” refers to a compound having the structure set forth below:

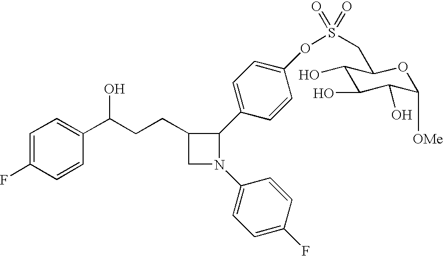

and stereoisomers thereof, and pharmaceutically acceptable salts and esters thereof.

Other useful exemplary ezetimibe derivatives, their synthesis and use are described, for example, in International Application Publication No. WO 2005/033100.

2. HMG-CoA Reductase Inhibitors

The pharmaceutical compositions and methods disclosed herein can further comprise one or more HMG CoA reductase inhibitors, also known as, “statins.” Statins slow down the body's production of cholesterol and inhibit the bioconversion of hydroxymethylglutaryl-coenzyme A to mevalonic acid catalyzed by the enzyme HMG-CoA reductase.

In one embodiment, the statin can include, atorvastatin (denoted as S1). As used herein, the term “atorvastatin” refers to a compound known in the art as atorvastatin (7-[2-(4-fluorophenyl)-5-(1-methylethyl)-3-phenyl-4-(phenylcarbamoyl)-1H-pyrrol-1-yl]-3,5-dihydroxy-heptanoic acid; brand name: Lipitor) and pharmaceutically acceptable salts and esters thereof.

In another embodiment, the statin may be fluvastatin (denoted as S2). As used herein, the term “fluvastatin” refers to a compound known in the art as fluvastatin (sodium 7-[3-(4-fluorophenyl)-1-propan-2-yl-indol-2-yl]-3,5-dihydroxy-hept-6-enoate; brand name: Lescol) and pharmaceutically acceptable salts and esters thereof.

In another embodiment, the statin may be lovastatin (denoted as S3). As used herein, the term “lovastatin” refers to a compound known in the art as lovastatin (8-[2-(4-hydroxy-6-oxo-tetrahydropyran-2-yl)ethyl]-3,7-dimethyl-1,2,3,7,8,8a-hexahydronaphthalen-1-yl]2-methylbutanoate; brand names: Altocor, Mevacor) and pharmaceutically acceptable salts and esters thereof.

In another embodiment, the statin may be pravastatin (denoted as S4). As used herein, the term “pravastatin” refers to a compound known in the art as pravastatin (5-dihydroxy-7-[6-hydroxy-2-methyl-8-(2-methylbutanoyloxy)-1,2,6,7,8,8a-hexahydronaphthalen-1-yl]-heptanoicacid; brand name: Pravachol) and pharmaceutically acceptable salts and esters thereof.

In another embodiment, the statin may be rosuvastatin (denoted as S5). As used herein, the term “rosuvastatin” refers to a compound known in the art as rosuvastatin (7-[4-(4-fluorophenyl)-6-(1-methylethyl)-2-(methyl-methylsulfonylamino)-pyrimidin-5-yl]-3,5-dihydroxy-hept-6-enoic acid; brand name: Crestor) and pharmaceutically acceptable salts and esters thereof.

In another embodiment, the statin may be pitavastatin (denoted as S6). As used herein, the term “pitavastatin” refers to a compound known in the art as pitavastatin and pharmaceutically acceptable salts and esters thereof.

In another embodiment, the statin may be tenivastatin (denoted as S7). As used herein, the term “tenivastatin” refers to a compound known in the art as tenivastatin and pharmaceutically acceptable salts and esters thereof.

In another embodiment, the statin may be simvastatin (denoted as S8). As used herein, the term “simvastatin” refers to a compound known in the art as simvastatin (7-(2,6-dimethyl-8-(2,2-dimethylbutyryloxy)-1,2,6,7,8,8a-hexahydro-1-naphthyl)-3,5-dihydroxyheptanoic acid; brand name: Zocor) and pharmaceutically acceptable salts and esters thereof.

In another embodiment, the statin may be rivastatin (denoted as S9). As used herein, the term “rivastatin” refers to a compound known in the art as rivastatin and pharmaceutically acceptable salts and esters thereof.

In another embodiment, the statin may be mevastatin (denoted as S10). As used herein, the term “mevastatin” refers to a compound known in the art as mevastatin and pharmaceutically acceptable salts and esters thereof.

In another embodiment, the statin may be cerivastatin (denoted as S11). As used herein, the term “cerivastatin” refers to a compound known in the art as cerivastatin and pharmaceutically acceptable salts and esters thereof.

3. Bile Acid Sequestrants

Bile acid sequestrants, also known as resins, help lower levels of LDL.

In one embodiment, the bile acid sequestrants may be cholestyramine (brand names: Locholest, Prevalite, Questran) (denoted as B1), including pharmaceutically acceptable salts and esters thereof.

In one embodiment, the bile acid sequestrants may be colesevelam (brand name: Welchol) (denoted as B2), including pharmaceutically acceptable salts and esters thereof.

In one embodiment, the bile acid sequestrants may be colestipol (brand name: Colestid) (denoted as B3), including pharmaceutically acceptable salts and esters thereof.

4. Fibrates

Fibrates (also known as fibric acid derivatives) help lower the cholesterol by reducing the amount of triglycerides (fats) in the body and by increasing the level of “good” cholesterol (also called HDL, or high-density lipoprotein).

In one embodiment, the fibrate may be fenofibrate (1-methylethyl2-[4-(4-chlorobenzoyl)-phenoxy]-2-methyl-propanoate; brand name: Tricor) (denoted as F1), including pharmaceutically acceptable salts and esters thereof.

In one embodiment, the fibrate may be bezafibrate (denoted as F2), including pharmaceutically acceptable salts and esters thereof.

In one embodiment, the fibrate may be ciprofibrate (denoted as F3), including pharmaceutically acceptable salts and esters thereof.

In one embodiment, the fibrate may be clofibrate (denoted as F4), including pharmaceutically acceptable salts and esters thereof.

In one embodiment, the fibrate may be gemfibrozil (5-(2,5-dimethylphenoxy)-2,2-dimethyl-pentanoicacid; brand name: Lopid) (denoted as F5), including pharmaceutically acceptable salts and esters thereof.

5. Niacin

It is contemplated that the composition may further comprise niacin (also called nicotinic acid), which is a B vitamin (denoted as N1). When given in large doses, niacin can lower the levels of triglycerides and LDL cholesterol, and increase the HDL cholesterol level.

6. Squalene Synthetase Inhibitors

Squalene synthetase inhibitors includes compounds which inhibit the condensation of molecules of farnesylpyrophosphate to form squalene, catalyzed by the enzyme squalene synthetase. Inhibition is readily determined by those skilled in the art according to standard assays (e.g., Meth. Enzymol, 15, 393-454 (1969) and Meth. Enzymol, 110, 359-373 (1985)). A variety of these compounds are known to those skilled in the art, e.g., in U.S. Pat. No. 5,026,554, disclosing fermentation products of the microorganism MF5465 (ATCC 74011) including zaragozic acid. A summary of other squalene synthetase inhibitors has been compiled (Curr. Op. Ther. Patents, 3, 861-4 (1993)).

In one embodiment, the squalene synthetase inhibitor may be TAK-475 (denoted as SQ1), including pharmaceutically acceptable salts and esters thereof.

In one embodiment, the squalene synthetase inhibitor may be ER-27856 (denoted as SQ2), including pharmaceutically acceptable salts and esters thereof.

In one embodiment, the squalene synthetase inhibitor may be RPR-107393 (denoted as SQ3), including pharmaceutically acceptable salts and esters thereof.

7. ACAT Inhibitors

ACAT inhibitors refer to compounds that inhibit the intracellular esterification of dietary cholesterol by the enzyme acyl CoA: cholesterol acyltransferase. Such inhibition may be determined readily by one of skill in the art according to standard assays, such as the method described in Heider et al., Journal of Lipid Research., 24,1127 (1983). A variety of these compounds are well known to those skilled in the art, e.g., U.S. Pat. No. 5,510,379 (carboxysulfonates), WO 96/26948 and WO 96/10559 (urea derivatives having ACAT inhibitory activity); DL-melinamide (GB Pat. No. 1,123,004 and Japan. J. Pharmacol., 42, 517-523 (1986); 2,2-dimethyl-N-(2,4,6 trimethoxyphenyl)dodecanamide (U.S. Pat. No. 4,716,175); and N-[2,6-bis(1methylethyl)phenyl]-N′-[[1-(4-dimethylaminophenyl)cyclopentyl]-methyl urea (U.S. Pat. No. 5,015,644).

In one embodiment, the ACAT inhibitor may be avasimibe (denoted as ACAT1), including pharmaceutically acceptable salts and esters thereof.

In one embodiment, the ACAT inhibitor may be pactimibe (denoted as ACAT2), uding pharmaceutically acceptable salts and esters thereof.

In one embodiment, the ACAT inhibitor may be HL-004 (denoted as ACAT3), including pharmaceutically acceptable salts and esters thereof.

8. CETP Inhibitors

CETP inhibitors includes compounds that inhibit the cholesterol ester transfer protein (CETP)-mediated transport of various cholesteryl esters and triglycerides from HDL to LDL and VLDL. Such CETP inhibition activity is readily determined by those skilled in the art according to standard assays (e.g., U.S. Pat. No. 6,140,343). A variety of CETP inhibitors will be known to those skilled in the art, including U.S. Pat. Nos. 6,140,343 (4-amino substituted-2-substituted-1,2,3,4 tetrahydroquinolines); 5,512,548 (polypeptide derivatives) and CETP-inhibitory rosenonolactone derivatives and phosphate-containing analogs of cholesteryl ester (J. ANTIBIOT., 49(8), 815-816 (1996), and BIOORG. MED. CHEM LETT., 6, 1951-1954 (1996), respectively.)

In one embodiment, the CETP inhibitor may be torcetrapib (denoted as CETP1), including pharmaceutically acceptable salts and esters thereof.

In one embodiment, the CETP inhibitor may be JTT-705 (denoted as CETP2), including pharmaceutically acceptable salts and esters thereof.

9. Other Classes of Compounds

Other classes of compounds that can be used in combination with a MTPI inhibitor includes PPAR (peroxisome proliferator activated receptor) alpha, delta, or gamma agonists such as muraglitazar, anti-inflammatory agents; LXR (liver X receptor), FXR, and RXR (retinoid X receptor) agonists; ABC (ATP binding cassette) transporters; and CB-1 (cannaboid)antagonists such as rimonabant (5-(4-Chlorophenyl)-1-(2,4-dichlorophenyl)-4-methyl-N-(piperidin-1-yl)-1H-pyrazole-3-carboxamide; Sanofi-Synthelab). Also contemplated compounds for use in the compositions and methods of this disclosure include ω-3 fatty acids, ileal bile acid co-transporters and inhibitors of same (IBATs), niacin receptor agonists, metformin, DPP-IV antagonists, sulphonylurea (SU), FAB protein inhibitors, and GLP-1 agonists. Further contemplated compounds that may be useful for co-therapy with MTPIs, especially for prevention of type 2 diabetes with for example insulin resistant include those compounds used for treating impaired glucose tolerance and/or impaired fasting tolerance.

Exemplary combinations of an MTP inhibitor, a CAI and a HMG-CoA reductase inhibitor are described in more detail in TABLE 1.

TABLE 1

| | | HMG-CoA |
|---|---|---|
| | | REDUCTASE |
| MTP INHIBITOR | CAI | INHIBITOR |
| M1 or M2 or M3 or | C1 (ezetimibe) | S1 (atorvastatin) |
| M4 or M5or M6 | | |
| M1 or M2 or M3 or | C1 (ezetimibe) | S2 (fluvastatin) |
| M4 or M5 or M6 | | |
| M1 or M2 or M3 or | C1 (ezetimibe) | S3 (lovastatin) |
| M4 or M5 or M6 | | |
| M1 or M2 or M3 or | C1 (ezetimibe) | S4 (pravastatin) |
| M4 or M5or M6 | | |
| M1 or M2 or M3 or | C1 (ezetimibe) | S5 (rosuvastatin |
| M4 or M5 or M6 | | |
| M1 or M2 or M3 or | C1 (ezetimibe) | S6 (pitavastatin) |
| M4 or M5 or M6 | | |
| M1 or M2 or M3 or | C1 (ezetimibe) | S7 (tenivastatin) |
| M4 or M5 or M6 | | |
| M1 or M2 or M3 or | C1 (ezetimibe) | S8 (simvastanin) |
| M4 or M5 or M6 | | |
| M1 or M2 or M3 or | C1 (ezetimibe) | S9 (rivastatin) |
| M4 or M5 or M6 | | |
| M1 or M2 or M3 or | C1 (ezetimibe) | S10 (mevastatin) |
| M4 or M5 or M6 | | |
| M1 or M2 or M3 or | C1 (ezetimibe) | S11 (cerivastatin) |
| M4 or M5 or M6 | | |
| M1 or M2 or M3 or | C2 (MD-0727) | S1 (atorvastatin) |
| M4 or M5 or M6 | | |
| M1 or M2 or M3 or | C2 (MD-0727) | S2 (fluvastatin) |
| M4 or M5 or M6 | | |
| M1 or M2 or M3 or | C2 (MD-0727) | S3 (lovastatin) |
| M4 or M5 or M6 | | |
| M1 or M2 or M3 or | C2 (MD-0727) | S4 (pravastatin) |
| M4 or M5 or M6 | | |
| M1 or M2 or M3 or | C2 (MD-0727) | S5 (rosuvastatin) |
| M4 or M5 or M6 | | |
| M1 or M2 or M3 or | C2 (MD-0727) | S6 (pitavastatin) |
| M4 or M5 or M6 | | |
| M1 or M2 or M3 or | C2 (MD-0727) | S7 (tenivastatin) |
| M4 or M5 or M6 | | |
| M1 or M2 or M3 or | C2 (MD-0727) | S8 (simvastanin) |
| M4 or M5 or M6 | | |
| M1 or M2 or M3 or | C2 (MD-0727) | S9 (rivastatin) |
| M4 or M5 or M6 | | |
| M1 or M2 or M3 or | C2 (MD-0727) | S10 (mevastatin) |
| M4 or M5 or M6 | | |
| M1 or M2 or M3 or | C2 (MD-0727) | S11 (cerivastatin) |
| M4 or M5 or M6 | | |
| M1 or M2 or M3 or | C3 (FM-VP4) | S1 (atorvastatin) |
| M4 or M5 or M6 | | |
| M1 or M2 or M3 or | C3 (FM-VP4) | S2 (fluvastatin) |
| M4 or M5 or M6 | | |
| M1 or M2 or M3 or | C3 (FM-VP4) | S3 (lovastatin) |
| M4 or M5 or M6 | | |
| M1 or M2 or M3 or | C3 (FM-VP4) | S4 (pravastatin) |
| M4 or M5 or M6 | | |
| M1 or M2 or M3 or | C3 (FM-VP4)) | S5 (rosuvastatin) |
| M4 or M5 or M6 | | |
| M1 or M2 or M3 or | C3 (FM-VP4) | S6 (pitavastatin) |
| M4 or M5 or M6 | | |
| M1 or M2 or M3 or | C3 (FM-VP4) | S7 (tenivastatin) |
| M4 or M5 or M6 | | |
| M1 or M2 or M3 or | C3 (FM-VP4) | S8 (simvastanin) |
| M4 or M5 or M6 | | |
| M1 or M2 or M3 or | C3 (FM-VP4) | S9 (rivastatin) |
| M4 or M5 or M6 | | |
| M1 or M2 or M3 or | C3 (FM-VP4) | S10 (mevastatin) |
| M4 or M5 or M6 | | |
| M1 or M2 or M3 or | C3 (FM-VP4) | S11 (cervastatin) |
| M4 or M5 or M6 | | |
| M1 or M2 or M3 or | C4 | S1 (atorvastatin) |
| M4 or M5 or M6 | | |
| M1 or M2 or M3 or | C4 | S2 (fluvastatin) |
| M4 or M5 or M6 | | |
| M1 or M2 or M3 or | C4 | S3 (lovastatin) |
| M4 or M5 or M6 | | |
| M1 or M2 or M3 or | C4 | S4 (pravastatin) |
| M4 or M5 or M6 | | |
| M1 or M2 or M3 or | C4 | S5 (rosuvastatin) |
| M4 or M5 or M6 | | |
| M1 or M2 or M3 or | C4 | S6 (pitavastatin) |
| M4 or M5 or M6 | | |
| M1 or M2 or M3 or | C4 | S7 (tenivastatin) |
| M4 or M5 or M6 | | |
| M1 or M2 or M3 or | C4 | S8 (simvastanin) |
| M4 or M5 or M6 | | |
| M1 or M2 or M3 or | C4 | S9 (rivastatin) |
| M4 or M5 or M6 | | |
| M1 or M2 or M3 or | C4 | S10 (mevastatin) |
| M4 or M5 or M6 | | |
| M1 or M2 or M3 or | C4 | S11 (cerivastatin) |
| M4 or M5 or M6 | | |
| M1 or M2 or M3 or | C5 (LPD179) | S1 (atorvastatin) |
| M4 or M5 or M6 | | |
| M1 or M2 or M3 or | C5 (LPD179) | S2 (fluvastatin) |
| M4 or M5 or M6 | | |
| M1 or M2 or M3 or | C5 (LPD179) | S3 (lovastatin) |
| M4 or M5 or M6 | | |
| M1 or M2 or M3 or | C5 (LPD179) | S4 (pravastatin) |
| M4 or M5 or M6 | | |
| M1 or M2 or M3 or | C5 (LPD179) | S5 (rosuvastatin) |
| M4 or M5 or M6 | | |
| M1 or M2 or M3 or | C5 (LPD179) | S6 (pitavastatin) |
| M4 or M5 or M6 | | |
| M1 or M2 or M3 or | C5 (LPD179) | S7 (tenivastatin) |
| M4 or M5 or M6 | | |
| M1 or M2 or M3 or | C5 (LPD179) | S8 (simvastanin) |
| M4 or M5 or M6 | | |
| M1 or M2 or M3 or | C5 (LPD179) | S9 (rivastatin) |
| M4 or M5 or M6 | | |
| M1 or M2 or M3 or | C5 (LPD179) | S10 (mevastatin) |
| M4 or M5 or M6 | | |
| M1 or M2 or M3 or | C5 (LPD179) | S11 (cerivastatin) |
| M4 or M5 or M6 | | |
| M1 or M2 or M3 or | C6 (LPD84) | S1 (atorvastatin) |
| M4 or M5 or M6 | | |
| M1 or M2 or M3 or | C6 (LPD84) | S2 (fluvastatin) |
| M4 or M5 or M6 | | |
| M1 or M2 or M3 or | C6 (LPD84) | S3 (lovastatin) |
| M4 or M5 or M6 | | |
| M1 or M2 or M3 or | C6 (LPD84) | S4 (pravastatin) |
| M4 or M5 or M6 | | |
| M1 or M2 or M3 or | C6 (LPD84) | S5 (rosuvastatin) |
| M4 or M5 or M6 | | |
| M1 or M2 or M3 or | C6 (LPD84) | S6 (pitavastatin) |
| M4 or M5 or M6 | | |
| M1 or M2 or M3 or | C6 (LPD84) | S7 (tenivastatin) |
| M4 or M5 or M6 | | |
| M1 or M2 or M3 or | C6 (LPD84) | S8 (simvastanin) |
| M4 or M5 or M6 | | |
| M1 or M2 or M3 or | C6 (LPD84) | S9 (rivastatin) |
| M4 or M5 or M6 | | |
| M1 or M2 or M3 or | C6 (LPD84) | S10 (mevastatin) |
| M4 or M5 or M6 | | |
| M1 or M2 or M3 or | C6 (LPD84) | S11 (cerivastatin) |
| M4 or M5 or M6 | | |
| M1 or M2 or M3 or | C7 (LPD145) | S1 (atorvastatin) |
| M4 or M5 or M6 | | |
| M1 or M2 or M3 or | C7 (LPD145)) | S2 (fluvastatin) |
| M4 or M5 or M6 | | |
| M1 or M2 or M3 or | C7 (LPD145) | S3 (lovastatin) |
| M4 or M5 or M6 | | |
| M1 or M2 or M3 or | C7 (LPD145)) | S4 (pravastatin) |
| M4 or M5 or M6 | | |
| M1 or M2 or M3 or | C7 (LPD145) | S5 (rosuvastatin) |
| M4 or M5 or M6 | | |
| M1 or M2 or M3 or | C7 (LPD145) | S6 (pitavastatin) |
| M4 or M5 or M6 | | |
| M1 or M2 or M3 or | C7 (LPD145) | S7 (tenivastatin) |
| M4 or M5 or M6 | | |
| M1 or M2 or M3 or | C7 (LPD145) | S8 (simvastanin) |
| M4 or M5 or M6 | | |
| M1 or M2 or M3 or | C7 (LPD145) | S9 (rivastatin) |
| M4 or M5 or M6 | | |
| M1 or M2 or M3 or | C7 (LPD145) | S10 (mevastatin) |
| M4 or M5 or M6 | | |
| M1 or M2 or M3 or | C7 (LPD145) | S11 (cerivastatin) |
| M4 or M5 or M6 | | |

It is understood that, as shown in TABLE 1, the MTP inhibitor present in each triple combination can be an MTP inhibitor denoted as M1 or M2 or M3 or M4 or M5 or M6.

Exemplary combinations of an MTP, a CAI and a fibric acid derivative are denoted in TABLE 2.

TABLE 2

| MTP INHIBITOR | CAI | FIBRATE |
|---|---|---|
| M1 or M2 or M3 or | C1 (ezetimibe) | F1 (fenofibrate) |
| M4 or M5 or M6 | | |
| M1 or M2 or M3 or | C1 (ezetimibe) | F2 (bezafibrate) |
| M4 or M5 or M6 | | |
| M1 or M2 or M3 or | C1 (ezetimibe) | F3 (ciprofibrate) |
| M4 or M5 or M6 | | |
| M1 or M2 or M3 or | C1 (ezetimibe) | F4 (clofibrate) |
| M4 or M5 or M6 | | |
| M1 or M2 or M3 or | C1 (ezetimibe) | F5 (gemfibrozil) |
| M4 or M5 or M6 | | |
| M1 or M2 or M3 or | C2 (MD-0727) | F1 (fenofibrate) |
| M4 or M5 or M6 | | |
| M1 or M2 or M3 or | C2 (MD-0727) | F2 (bezafibrate) |
| M4 or M5 or M6 | | |
| M1 or M2 or M3 or | C2 (MD-0727) | F3 (ciprofibrate) |
| M4 or M5 or M6 | | |
| M1 or M2 or M3 or | C2 (MD-0727) | F4 (clofibrate) |
| M4 or M5 or M6 | | |
| M1 or M2 or M3 or | C2 (MD-0727) | F5 (gemfibrozil) |
| M4 or M5 or M6 | | |
| M1 or M2 or M3 or | C3 (FM-VP4) | F1 (fenofibrate) |
| M4 or M5 or M6 | | |
| M1 or M2 or M3 or | C3 (FM-VP4) | F2 (bezafibrate) |
| M4 or M5 or M6 | | |
| M1 or M2 or M3 or | C3 (FM-VP4) | F3 (ciprofibrate) |
| M4 or M5 or M6 | | |
| M1 or M2 or M3 or | C3 (FM-VP4) | F4 (clofibrate) |
| M4 or M5 or M6 | | |
| M1 or M2 or M3 or | C3 (FM-VP4) | F5 (gemfibrozil) |
| M4 or M5 or M6 | | |
| M1 or M2 or M3 or | C4 | F1 (fenofibrate) |
| M4 or M5 or M6 | | |
| M1 or M2 or M3 or | C4 | F2 (bezafibrate) |
| M4 or M5 or M6 | | |
| M1 or M2 or M3 or | C4 | F3 (ciprofibrate) |
| M4 or M5 or M6 | | |
| M1 or M2 or M3 or | C4 | F4 (clofibrate) |
| M4 or M5 or M6 | | |
| M1 or M2 or M3 or | C4 | F5 (gemfibrozil) |
| M4 or M5 or M6 | | |
| M1 or M2 or M3 or | C5 (LPD179) | F1 (fenofibrate) |
| M4 or M5 or M6 | | |
| M1 or M2 or M3 or | C5 (LPD179) | F2 (bezafibrate) |
| M4 or M5 or M6 | | |
| M1 or M2 or M3 or | C5 (LPD179) | F3 (ciprofibrate) |
| M4 or M5 or M6 | | |
| M1 or M2 or M3 or | C5 (LPD179) | F4 (clofibrate) |
| M4 or M5 or M6 | | |
| M1 or M2 or M3 or | C5 (LPD179) | F5 (gemfibrozil) |
| M4 or M5 or M6 | | |
| M1 or M2 or M3 or | C6 (LPD84) | F1 (fenofibrate) |
| M4 or M5 or M6 | | |
| M1 or M2 or M3 or | C6 (LPD84) | F2 (bezafibrate) |
| M4 or M5 or M6 | | |
| M1 or M2 or M3 or | C6 (LPD84) | F3 (ciprofibrate) |
| M4 or M5 or M6 | | |
| M1 or M2 or M3 or | C6 (LPD84) | F4 (clofibrate) |
| M4 or M5 or M6 | | |
| M1 or M2 or M3 or | C6 (LPD84) | F5 (gemfibrozil) |
| M4 or M5 or M6 | | |
| M1 or M2 or M3 or | C7 (LPD145) | F1 (fenofibrate) |
| M4 or M5 or M6 | | |
| M1 or M2 or M3 or | C7 (LPD145) | F2 (bezafibrate) |
| M4 or M5 or M6 | | |
| M1 or M2 or M3 or | C7 (LPD145) | F3 (ciprofibrate) |
| M4 or M5 or M6 | | |
| M1 or M2 or M3 or | C7 (LPD145) | F4 (clofibrate) |
| M4 or M5 or M6 | | |
| M1 or M2 or M3 or | C7 (LPD145) | F5 (gemfibrozil) |
| M4 or M5 or M6 | | |

It is understood that, as shown in TABLE 2, the MTP inhibitor present in each triple combination can be an MTP inhibitor denoted as M1 or M2 or M3 or M4 or M5 or M6.

Exemplary combinations of an MTP inhibitor, a CAI and a niacin are denoted in TABLE 3.

TABLE 3

| MTP INHIBITOR | CAI | NIACIN |
|---|---|---|
| M1 or M2 or M3 or | C1 (ezetimibe) | N1 (niacin) |
| M4 or M5 or M6 | | |
| M1 or M2 or M3 or | C2 (MD-0727) | N1 (niacin) |
| M4 or M5 or M6 | | |
| M1 or M2 or M3 or | C3 (FM-VP4) | N1 (niacin) |
| M4 or M5 or M6 | | |
| M1 or M2 or M3 or | C4 | N1 (niacin) |
| M4 or M5 or M6 | | |
| M1 or M2 or M3 or | C5 (LPD179) | N1 (niacin) |
| M4 or M5 or M6 | | |
| M1 or M2 or M3 or | C6 (LPD84) | N1 (niacin) |
| M4 or M5 or M6 | | |
| M1 or M2 or M3 or | C7 (LPD-145) | N1 (niacin) |
| M4 or M5 or M6 | | |

It is understood that, as shown in TABLE 3, the MTP inhibitor present in each triple combination can be an MTP inhibitor denoted as M1 or M2 or M3 or M4 or M5 or M6.

Exemplary combinations of an MTP inhibitor, a HMG-CoA reductase inhibitor and a fibric acid derivative are denoted in TABLE 4.

TABLE 4

| | HMG-CoA | |
|---|---|---|
| | REDUCTASE | |
| MTP INHIBITOR | INHIBITOR | FIBRATE |
| M1 or M2 or M3 or | S1 (atorvastatin) | F1 (fenofibrate) |
| M4 or M5 or M6 | | |
| M1 or M2 or M3 or | S2 (fluvastatin) | F1 (fenofibrate) |
| M4 or M5 or M6 | | |
| M1 or M2 or M3 or | S3 (lovastatin) | F1 (fenofibrate) |
| M4 or M5 or M6 | | |
| M1 or M2 or M3 or | S4 (pravastatin) | F1 (fenofibrate) |
| M4 or M5 or M6 | | |
| M1 or M2 or M3 or | S5 (rosuvastatin) | F1 (fenofibrate) |
| M4 or M5 or M6 | | |
| M1 or M2 or M3 or | S6 (pitavastatin) | F1 (fenofibrate) |
| M4 or M5 or M6 | | |
| M1 or M2 or M3 or | S7 (tenivastatin) | F1 (fenofibrate) |
| M4 or M5 or M6 | | |
| M1 or M2 or M3 or | S8 (simvastanin) | F1 (fenofibrate) |
| M4 or M5 or M6 | | |
| M1 or M2 or M3 or | S9 (rivastatin) | F1 (fenofibrate) |
| M4 or M5 or M6 | | |
| M1 or M2 or M3 or | S10 (mevastatin) | F1 (fenofibrate) |
| M4 or M5 or M6 | | |
| M1 or M2 or M3 or | S11 (cerivastatin) | F1 (fenofibrate) |
| M4 or M5 or M6 | | |
| M1 or M2 or M3 or | S1 (atorvastatin) | F2 (bezafibrate) |
| M4 or M5 or M6 | | |
| M1 or M2 or M3 or | S2 (fluvastatin) | F2 (bezafibrate) |
| M4 or M5 or M6 | | |
| M1 or M2 or M3 or | S3 (lovastatin) | F2 (bezafibrate) |
| M4 or M5 or M6 | | |
| M1 or M2 or M3 or | S4 (pravastatin) | F2 (bezafibrate) |
| M4 or M5 or M6 | | |
| M1 or M2 or M3 or | S5 (rosuvastatin) | F2 (bezafibrate) |
| M4 or M5 or M6 | | |
| M1 or M2 or M3 or | S6 (pitavastatin) | F2 (bezafibrate) |
| M4 or M5 or M6 | | |
| M1 or M2 or M3 or | S7 (tenivastatin) | F2 (bezafibrate) |
| M4 or M5 or M6 | | |
| M1 or M2 or M3 or | S8 (simvastanin) | F2 (bezafibrate) |
| M4 or M5 or M6 | | |
| M1 or M2 or M3 or | S9 (rivastatin) | F2 (bezafibrate) |
| M4 or M5 or M6 | | |
| M1 or M2 or M3 or | S10 (mevastatin) | F2 (bezafibrate) |
| M4 or M5 or M6 | | |
| M1 or M2 or M3 or | S11 (cerivastatin) | F2 (bezafibrate) |
| M4 or M5 or M6 | | |
| M1 or M2 or M3 or | S1 (atorvastatin) | F3 (ciprofibrate) |
| M4 or M5 or M6 | | |
| M1 or M2 or M3 or | S2 (fluvastatin) | F3 (ciprofibrate) |
| M4 or M5 or M6 | | |
| M1 or M2 or M3 or | S3 (lovastatin) | F3 (ciprofibrate) |
| M4 or M5 or M6 | | |
| M1 or M2 or M3 or | S4 (pravastatin) | F3 (ciprofibrate) |
| M4 or M5 or M6 | | |
| M1 or M2 or M3 or | S5 (rosuvastatin) | F3 (ciprofibrate) |
| M4 or M5 or M6 | | |
| M1 or M2 or M3 or | S6 (pitavastatin) | F3 (ciprofibrate) |
| M4 or M5 or M6 | | |
| M1 or M2 or M3 or | S7 (tenivastatin) | F3 (ciprofibrate) |
| M4 or M5 or M6 | | |
| M1 or M2 or M3 or | S8 (simvastanin) | F3 (ciprofibrate) |
| M4 or M5 or M6 | | |
| M1 or M2 or M3 or | S9 (rivastatin) | F3 (ciprofibrate) |
| M4 or M5 or M6 | | |
| M1 or M2 or M3 or | S10 (mevastatin) | F3 (ciprofibrate) |
| M4 or M5 or M6 | | |
| M1 or M2 or M3 or | S11 (cerivastatin) | F3 (ciprofibrate) |
| M4 or M5 or M6 | | |
| M1 or M2 or M3 or | S1 (atorvastatin) | F4 (clofibrate) |
| M4 or M5 or M6 | | |
| M1 or M2 or M3 or | S2 (fluvastatin) | F4 (clofibrate) |
| M4 or M5 or M6 | | |
| M1 or M2 or M3 or | S3 (lovastatin) | F4 (clofibrate) |
| M4 or M5 or M6 | | |
| M1 or M2 or M3 or | S4 (pravastatin) | F4 (clofibrate) |
| M4 or M5 or M6 | | |
| M1 or M2 or M3 or | S5 (rosuvastatin) | F4 (clofibrate) |
| M4 or M5 or M6 | | |
| M1 or M2 or M3 or | S6 (pitavastatin) | F4 (clofibrate) |
| M4 or M5 or M6 | | |
| M1 or M2 or M3 or | S7 (tenivastatin) | F4 (clofibrate) |
| M4 or M5 or M6 | | |
| M1 or M2 or M3 or | S8 (simvastanin) | F4 (clofibrate) |
| M4 or M5 or M6 | | |
| M1 or M2 or M3 or | S9 (rivastatin) | F4 (clofibrate) |
| M4 or M5 or M6 | | |
| M1 or M2 or M3 or | S10 (mevastatin) | F4 (clofibrate) |
| M4 or M5 or M6 | | |
| M1 or M2 or M3 or | S11 (cerivastatin) | F4 (clofibrate) |
| M4 or M5 or M6 | | |
| M1 or M2 or M3 or | S1 (atorvastatin) | F5 (gemfibrozil) |
| M4 or M5 or M6 | | |
| M1 or M2 or M3 or | S2 (fluvastatin) | F5 (gemfibrozil) |
| M4 or M5 or M6 | | |
| M1 or M2 or M3 or | S3 (lovastatin) | F5 (gemfibrozil) |
| M4 or M5 or M6 | | |
| M1 or M2 or M3 or | S4 (pravastatin) | F5 (gemfibrozil) |
| M4 or M5 or M6 | | |
| M1 or M2 or M3 or | S5 (rosuvastatin) | F5 (gemfibrozil) |
| M4 or M5 or M6 | | |
| M1 or M2 or M3 or | S6 (pitavastatin) | F5 (gemfibrozil) |
| M4 or M5 or M6 | | |
| M1 or M2 or M3 or | S7 (tenivastatin) | F5 (gemfibrozil) |
| M4 or M5 or M6 | | |
| M1 or M2 or M3 or | S8 (simvastanin) | F5 (gemfibrozil) |
| M4 or M5 or M6 | | |
| M1 or M2 or M3 or | S9 (rivastatin) | F5 (gemfibrozil) |
| M4 or M5 or M6 | | |
| M1 or M2 or M3 or | S10 (mevastatin) | F5 (gemfibrozil) |
| M4 or M5 or M6 | | |
| M1 or M2 or M3 or | S11 (cerivastatin) | F5 (gemfibrozil) |
| M4 or M5 or M6 | | |

It is understood that, as shown in TABLE 4, the MTP inhibitor present in each triple combination can be an MTP inhibitor denoted as M1 or M2 or M3 or M4 or M5 or M6. It is contemplated that this combination of active ingredients will be particularly effective at reducing the levels of triglycerides in the blood and, therefore, will be helpful in treating hypertriglyceridemias. As a result, these combinations will be helpful in reducing the risk of pancreatitis in patients with elevated levels of triglycerides in the blood.

Exemplary combinations of an MTP inhibitor, a HMG-CoA reductase inhibitor and a niacin are denoted in TABLE 5.

TABLE 5

| | HMG-CoA | |
|---|---|---|
| | REDUCTASE | |
| MTP INHIBITOR | INHIBITOR | NIACIN |
| M1 or M2 or M3 or | S1 (atorvastatin) | N1 (niacin) |
| M4 or M5 or M6 | | |
| M1 or M2 or M3 or | S2 (fluvastatin) | N1 (niacin) |
| M4 or M5 or M6 | | |
| M1 or M2 or M3 or | S3 (lovastatin) | N1 (niacin) |
| M4 or M5 or M6 | | |
| M1 or M2 or M3 or | S4 (pravastatin) | N1 (niacin) |
| M4 or M5 or M6 | | |
| M1 or M2 or M3 or | S5 (rosuvastatin) | N1 (niacin) |
| M4 or M5 or M6 | | |
| M1 or M2 or M3 or | S6 (pitavastatin) | N1 (niacin) |
| M4 or M5 or M6 | | |
| M1 or M2 or M3 or | S7 (tenivastatin) | N1 (niacin) |
| M4 or M5 or M6 | | |
| M1 or M2 or M3 or | S8 (simvastanin) | N1 (niacin) |
| M4 or M5 or M6 | | |
| M1 or M2 or M3 or | S9 (rivastatin) | N1 (niacin) |
| M4 or M5 or M6 | | |
| M1 or M2 or M3 or | S10 (mevastatin) | N1 (niacin) |
| M4 or M5 or M6 | | |
| M1 or M2 or M3 or | S11 (cerivastatin) | N1 (niacin) |
| M4 or M5 or M6 | | |

It is understood that, as shown in TABLE 5, the MTP inhibitor present in each triple combination can be an MTP inhibitor denoted as M1 or M2 or M3 or M4 or M5 or M6.

Exemplary combinations of an MTP inhibitor, a CAI and a squalene synthetase inhibitor are denoted in TABLE 6.

TABLE 6

| | | SQUALENE |
|---|---|---|
| | | SYNTHETASE |
| MTP INHIBITOR | CAI | INHIBITOR |
| M1 or M2 or M3 or | C1 (ezetimibe) | SQ1 (TAK-475) |
| M4 or M5 or M6 | | |
| M1 or M2 or M3 or | C2 (MD-0727) | SQ1 (TAK-475) |
| M4 or M5 or M6 | | |
| M1 or M2 or M3 or | C3 (FM-VP4) | SQ1 (TAK-475) |
| M4 or M5 or M6 | | |
| M1 or M2 or M3 or | C4 | SQ1 (TAK-475) |
| M4 or M5 or M6 | | |
| M1 or M2 or M3 or | C5 (LPD179) | SQ1 (TAK-475) |
| M4 or M5 or M6 | | |
| M1 or M2 or M3 or | C6 (LPD84) | SQ1 (TAK-475) |
| M4 or M5 or M6 | | |
| M1 or M2 or M3 or | C7 (LPD-145) | SQ1 (TAK-475) |
| M4 or M5 or M6 | | |
| M1 or M2 or M3 or | C1 (ezetimibe) | SQ2 (ER-27856) |
| M4 or M5 or M6 | | |
| M1 or M2 or M3 or | C2 (MD-0727) | SQ2 (ER-27856) |
| M4 or M5 or M6 | | |
| M1 or M2 or M3 or | C3 (FM-VP4) | SQ2 (ER-27856) |
| M4 or M5 or M6 | | |
| M1 or M2 or M3 or | C4 | SQ2 (ER-27856) |
| M4 or M5 or M6 | | |
| M1 or M2 or M3 or | C5 (LPD179) | SQ2 (ER-27856) |
| M4 or M5 or M6 | | |
| M1 or M2 or M3 or | C6 (LPD84) | SQ2 (ER-27856) |
| M4 or M5 or M6 | | |
| M1 or M2 or M3 or | C7 (LPD-145) | SQ2 (ER-27856) |
| M4 or M5 or M6 | | |
| M1 or M2 or M3 or | C1 (ezetimibe) | SQ3 (RPR-107393) |
| M4 or M5 or M6 | | |
| M1 or M2 or M3 or | C2 (MD-0727) | SQ3 (RPR-107393) |
| M4 or M5 or M6 | | |
| M1 or M2 or M3 or | C3 (FM-VP4) | SQ3 (RPR-107393) |
| M4 or M5 or M6 | | |
| M1 or M2 or M3 or | C4 | SQ3 (RPR-107393) |
| M4 or M5 or M6 | | |
| M1 or M2 or M3 or | C5 (LPD179) | SQ3 (RPR-107393) |
| M4 or M5 or M6 | | |
| M1 or M2 or M3 or | C6 (LPD84) | SQ3 (RPR-107393) |
| M4 or M5 or M6 | | |
| M1 or M2 or M3 or | C7 (LPD-145) | SQ3 (RPR-107393) |
| M4 or M5 or M6 | | |

It is understood that, as shown in TABLE 6, the MTP inhibitor present in each triple combination can be an MTP inhibitor denoted as M1 or M2 or M3 or M4 or M5 or M6.

Exemplary combinations of an MTP inhibitor, a CAI and an ACAT inhibitor are denoted in TABLE 7.

TABLE 7

| MTP INHIBITOR | CAI | ACAT INHIBITOR |
|---|---|---|
| M1 or M2 or M3 or | C1 (ezetimibe) | ACAT1 (avasimibe) |
| M4 or M5 or M6 | | |
| M1 or M2 or M3 or | C2 (MD-0727) | ACAT1 (avasimibe) |
| M4 or M5 or M6 | | |
| M1 or M2 or M3 or | C3 (FM-VP4) | ACAT1 (avasimibe) |
| M4 or M5 or M6 | | |
| M1 or M2 or M3 or | C4 | ACAT1 (avasimibe) |
| M4 or M5 or M6 | | |
| M1 or M2 or M3 or | C5 (LPD179) | ACAT1 (avasimibe) |
| M4 or M5 or M6 | | |
| M1 or M2 or M3 or | C6 (LPD84) | ACAT1 (avasimibe) |
| M4 or M5 or M6 | | |
| M1 or M2 or M3 or | C7 (LPD-145) | ACAT1 (avasimibe) |
| M4 or M5 or M6 | | |
| M1 or M2 or M3 or | C1 (ezetimibe) | ACAT2 (pactimibe) |
| M4 or M5 or M6 | | |
| M1 or M2 or M3 or | C2 (MD-0727) | ACAT2 (pactimibe) |
| M4 or M5 or M6 | | |
| M1 or M2 or M3 or | C3 (FM-VP4) | ACAT2 (pactimibe) |
| M4 or M5 or M6 | | |
| M1 or M2 or M3 or | C4 | ACAT2 (pactimibe) |
| M4 or M5 or M6 | | |
| M1 or M2 or M3 or | C5 (LPD179) | ACAT2 (pactimibe) |
| M4 or M5 or M6 | | |
| M1 or M2 or M3 or | C6 (LPD84) | ACAT2 (pactimibe) |
| M4 or M5 or M6 | | |
| M1 or M2 or M3 or | C7 (LPD-145) | ACAT2 (pactimibe) |
| M4 or M5 or M6 | | |
| M1 or M2 or M3 or | C1 (ezetimibe) | ACAT3 (HL-004) |
| M4 or M5 or M6 | | |
| M1 or M2 or M3 or | C2 (MD-0727) | ACAT3 (HL-004) |
| M4 or M5 or M6 | | |
| M1 or M2 or M3 or | C3 (FM-VP4) | ACAT3 (HL-004) |
| M4 or M5 or M6 | | |
| M1 or M2 or M3 or | C4 | ACAT3 (HL-004) |
| M4 or M5 or M6 | | |
| M1 or M2 or M3 or | C5 (LPD179) | ACAT3 (HL-004) |
| M4 or M5 or M6 | | |
| M1 or M2 or M3 or | C6 (LPD84) | ACAT3 (HL-004) |
| M4 or M5 or M6 | | |
| M1 or M2 or M3 or | C7 (LPD-145) | ACAT3 (HL-004) |
| M4 or M5 or M6 | | |

It is understood that, as shown in TABLE 7, the MTP inhibitor present in each triple combination can be an MTP inhibitor denoted as M1 or M2 or M3 or M4 or M5 or M6.

Exemplary combinations of an MTP inhibitor, a CAI and a CETP inhibitor are denoted in TABLE 8.

TABLE 8

| MTP INHIBITOR | CAI | CETP INHIBITOR |
|---|---|---|
| M1 or M2 or M3 or | C1 (ezetimibe) | CETP1 (torcetrapib) |
| M4 or M5 or M6 | | |
| M1 or M2 or M3 or | C2 (MD-0727) | CETP1 (torcetrapib) |
| M4 or M5 or M6 | | |
| M1 or M2 or M3 or | C3 (FM-VP4) | CETP1 (torcetrapib) |
| M4 or M5 or M6 | | |
| M1 or M2 or M3 or | C4 | CETP1 (torcetrapib) |
| M4 or M5 or M6 | | |
| M1 or M2 or M3 or | C5 (LPD179) | CETP1 (torcetrapib) |
| M4 or M5 or M6 | | |
| M1 or M2 or M3 or | C6 (LPD84) | CETP1 (torcetrapib) |
| M4 or M5 or M6 | | |
| M1 or M2 or M3 or | C7 (LPD-145) | CETP1 (torcetrapib) |
| M4 or M5 or M6 | | |
| M1 or M2 or M3 or | C1 (ezetimibe) | CETP2 (JTT-705) |
| M4 or M5 or M6 | | |
| M1 or M2 or M3 or | C2 (MD-0727) | CETP2 (JTT-705) |
| M4 or M5 or M6 | | |
| M1 or M2 or M3 or | C3 (FM-VP4) | CETP2 (JTT-705) |
| M4 or M5 or M6 | | |
| M1 or M2 or M3 or | C4 | CETP2 (JTT-705) |
| M4 or M5 or M6 | | |
| M1 or M2 or M3 or | C5 (LPD179) | CETP2 (JTT-705) |
| M4 or M5 or M6 | | |
| M1 or M2 or M3 or | C6 (LPD84) | CETP2 (JTT-705) |
| M4 or M5 or M6 | | |
| M1 or M2 or M3 or | C7 (LPD-145) | CETP2 (JTT-705) |
| M4 or M5 or M6 | | |

It is understood that, as shown in TABLE 8, the MTP inhibitor present in each triple combination can be an MTP inhibitor denoted as M1 or M2 or M3 or M4 or M5 or M6.

Exemplary combinations of an MTP inhibitor, a HMG-CoA reductase inhibitor and a squalene synthetase inhibitor are denoted in TABLE 9.

TABLE 9

| | HMG-CoA | SQUALENE |
|---|---|---|
| | REDUCTASE | SYNTHETASE |
| MTP INHIBITOR | INHIBITOR | INHIBITOR |
| M1 or M2 or M3 or | S1 (atorvastatin) | SQ1 (TAK-475) |
| M4 or M5 or M6 | | |
| M1 or M2 or M3 or | S2 (fluvastatin) | SQ1 (TAK-475) |
| M4 or M5 or M6 | | |
| M1 or M2 or M3 or | S3 (lovastatin) | SQ1 (TAK-475) |
| M4 or M5 or M6 | | |
| M1 or M2 or M3 or | S4 (pravastatin) | SQ1 (TAK-475) |
| M4 or M5 or M6 | | |
| M1 or M2 or M3 or | S5 (rosuvastatin) | SQ1 (TAK-475) |
| M4 or M5 or M6 | | |
| M1 or M2 or M3 or | S6 (pitavastatin) | SQ1 (TAK-475) |
| M4 or M5 or M6 | | |
| M1 or M2 or M3 or | S7 (tenivastatin) | SQ1 (TAK-475) |
| M4 or M5 or M6 | | |
| M1 or M2 or M3 or | S8 (simvastanin) | SQ1 (TAK-475) |
| M4 or M5 or M6 | | |
| M1 or M2 or M3 or | S9 (rivastatin) | SQ1 (TAK-475) |
| M4 or M5 or M6 | | |
| M1 or M2 or M3 or | S10 (mevastatin) | SQ1 (TAK-475) |
| M4 or M5 or M6 | | |
| M1 or M2 or M3 or | S11 (cerivastatin) | SQ1 (TAK-475) |
| M4 or M5 or M6 | | |
| M1 or M2 or M3 or | S1 (atorvastatin) | SQ2 (ER-27856) |
| M4 or M5 or M6 | | |
| M1 or M2 or M3 or | S2 (fluvastatin) | SQ2 (ER-27856) |
| M4 or M5 or M6 | | |
| M1 or M2 or M3 or | S3 (lovastatin) | SQ2 (ER-27856) |
| M4 or M5 or M6 | | |
| M1 or M2 or M3 or | S4 (pravastatin) | SQ2 (ER-27856) |
| M4 or M5 or M6 | | |
| M1 or M2 or M3 or | S5 (rosuvastatin) | SQ2 (ER-27856) |
| M4 or M5 or M6 | | |
| M1 or M2 or M3 or | S6 (pitavastatin) | SQ2 (ER-27856) |
| M4 or M5 or M6 | | |
| M1 or M2 or M3 or | S7 (tenivastatin) | SQ2 (ER-27856) |
| M4 or M5 or M6 | | |
| M1 or M2 or M3 or | S8 (simvastanin) | SQ2 (ER-27856) |
| M4 or M5 or M6 | | |
| M1 or M2 or M3 or | S9 (rivastatin) | SQ2 (ER-27856) |
| M4 or M5 or M6 | | |
| M1 or M2 or M3 or | S10 (mevastatin) | SQ2 (ER-27856) |
| M4 or M5 or M6 | | |
| M1 or M2 or M3 or | S11 (cerivastatin) | SQ2 (ER-27856) |
| M4 or M5 or M6 | | |
| M1 or M2 or M3 or | S1 (atorvastatin) | SQ3 (RPR-107393) |
| M4 or M5 or M6 | | |
| M1 or M2 or M3 or | S2 (fluvastatin) | SQ3 (RPR-107393) |
| M4 or M5 or M6 | | |
| M1 or M2 or M3 or | S3 (lovastatin) | SQ3 (RPR-107393) |
| M4 or M5 or M6 | | |
| M1 or M2 or M3 or | S4 (pravastatin) | SQ3 (RPR-107393) |
| M4 or M5 or M6 | | |
| M1 or M2 or M3 or | S5 (rosuvastatin) | SQ3 (RPR-107393) |
| M4 or M5 or M6 | | |
| M1 or M2 or M3 or | S6 (pitavastatin) | SQ3 (RPR-107393) |
| M4 or M5 or M6 | | |
| M1 or M2 or M3 or | S7 (tenivastatin) | SQ3 (RPR-107393) |
| M4 or M5 or M6 | | |
| M1 or M2 or M3 or | S8 (simvastanin) | SQ3 (RPR-107393) |
| M4 or M5 or M6 | | |
| M1 or M2 or M3 or | S9 (rivastatin) | SQ3 (RPR-107393) |
| M4 or M5 or M6 | | |
| M1 or M2 or M3 or | S10 (mevastatin) | SQ3 (RPR-107393) |
| M4 or M5 or M6 | | |
| M1 or M2 or M3 or | S11 (cerivastatin) | SQ3 (RPR-107393) |
| M4 or M5 or M6 | | |

It is understood that, as shown in TABLE 9, the MTP inhibitor present in each triple combination can be an MTP inhibitor denoted as M1 or M2 or M3 or M4 or M5 or M6.

Exemplary combinations of an MTP inhibitor, a HMG-CoA reductase inhibitor and an ACAT inhibitor are denoted in TABLE 10.

TABLE 10

| | HMG-CoA | |
|---|---|---|
| | REDUCTASE | |
| MTP INHIBITOR | INHIBITOR | ACAT INHIBITOR |
| M1 or M2 or M3 or | S1 (atorvastatin) | ACAT1 (avasimibe) |
| M4 or M5 or M6 | | |
| M1 or M2 or M3 or | S2 (fluvastatin) | ACAT1 (avasimibe) |
| M4 or M5 or M6 | | |
| M1 or M2 or M3 or | S3 (lovastatin) | ACAT1 (avasimibe) |
| M4 or M5 or M6 | | |
| M1 or M2 or M3 or | S4 (pravastatin) | ACAT1 (avasimibe) |
| M4 or M5 or M6 | | |
| M1 or M2 or M3 or | S5 (rosuvastatin) | ACAT1 (avasimibe) |
| M4 or M5 or M6 | | |
| M1 or M2 or M3 or | S6 (pitavastatin) | ACAT1 (avasimibe) |
| M4 or M5 or M6 | | |
| M1 or M2 or M3 or | S7 (tenivastatin) | ACAT1 (avasimibe) |
| M4 or M5 or M6 | | |
| M1 or M2 or M3 or | S8 (simvastanin) | ACAT1 (avasimibe) |
| M4 or M5 or M6 | | |
| M1 or M2 or M3 or | S9 (rivastatin) | ACAT1 (avasimibe) |
| M4 or M5 or M6 | | |
| M1 or M2 or M3 or | S10 (mevastatin) | ACAT1 (avasimibe) |
| M4 or M5 or M6 | | |
| M1 or M2 or M3 or | S11 (cerivastatin) | ACAT1 (avasimibe) |
| M4 or M5 or M6 | | |
| M1 or M2 or M3 or | S1 (atorvastatin) | ACAT2 (pactimibe) |
| M4 or M5 or M6 | | |
| M1 or M2 or M3 or | S2 (fluvastatin) | ACAT2 (pactimibe) |
| M4 or M5 or M6 | | |
| M1 or M2 or M3 or | S3 (lovastatin) | ACAT2 (pactimibe) |
| M4 or M5 or M6 | | |
| M1 or M2 or M3 or | S4 (pravastatin) | ACAT2 (pactimibe) |
| M4 or M5 or M6 | | |
| M1 or M2 or M3 or | S5 (rosuvastatin) | ACAT2 (pactimibe) |
| M4 or M5 or M6 | | |
| M1 or M2 or M3 or | S6 (pitavastatin) | ACAT2 (pactimibe) |
| M4 or M5 or M6 | | |
| M1 or M2 or M3 or | S7 (tenivastatin) | ACAT2 (pactimibe) |
| M4 or M5 or M6 | | |
| M1 or M2 or M3 or | S8 (simvastanin) | ACAT2 (pactimibe) |
| M4 or M5 or M6 | | |
| M1 or M2 or M3 or | S9 (rivastatin) | ACAT2 (pactimibe) |
| M4 or M5 or M6 | | |
| M1 or M2 or M3 or | S10 (mevastatin) | ACAT2 (pactimibe) |
| M4 or M5 or M6 | | |
| M1 or M2 or M3 or | S11 (cerivastatin) | ACAT2 (pactimibe) |
| M4 or M5 or M6 | | |
| M1 or M2 or M3 or | S1 (atorvastatin) | ACAT3 (HL-004) |
| M4 or M5 or M6 | | |
| M1 or M2 or M3 or | S2 (fluvastatin) | ACAT3 (HL-004) |
| M4 or M5 or M6 | | |
| M1 or M2 or M3 or | S3 (lovastatin) | ACAT3 (HL-004) |
| M4 or M5 or M6 | | |
| M1 or M2 or M3 or | S4 (pravastatin) | ACAT3 (HL-004) |
| M4 or M5 or M6 | | |
| M1 or M2 or M3 or | S5 (rosuvastatin) | ACAT3 (HL-004) |
| M4 or M5 or M6 | | |
| M1 or M2 or M3 or | S6 (pitavastatin) | ACAT3 (HL-004) |
| M4 or M5 or M6 | | |
| M1 or M2 or M3 or | S7 (tenivastatin) | ACAT3 (HL-004) |
| M4 or M5 or M6 | | |
| M1 or M2 or M3 or | S8 (simvastanin) | ACAT3 (HL-004) |
| M4 or M5 or M6 | | |
| M1 or M2 or M3 or | S9 (rivastatin) | ACAT3 (HL-004) |
| M4 or M5 or M6 | | |
| M1 or M2 or M3 or | S10 (mevastatin) | ACAT3 (HL-004) |
| M4 or M5 or M6 | | |
| M1 or M2 or M3 or | S11 (cerivastatin) | ACAT3 (HL-004) |
| M4 or M5 or M6 | | |

It is understood that, as shown in TABLE 10, the MTP inhibitor present in each triple combination can be an MTP inhibitor denoted as M1 or M2 or M3 or M4 or M5 or M6.

Exemplary combinations of an MTP inhibitor, a HMG-CoA reductase inhibitor and a CETP inhibitor are denoted in TABLE 11.

TABLE 11

| | HMG-CoA | |
|---|---|---|
| | REDUCTASE | |
| MTP INHIBITOR | INHIBITOR | CETP INHIBITOR |
| M1 or M2 or M3 or | S1 (atorvastatin) | CETP1 (torcetrapib) |
| M4 or M5 or M6 | | |
| M1 or M2 or M3 or | S2 (fluvastatin) | CETP1 (torcetrapib) |
| M4 or M5 or M6 | | |
| M1 or M2 or M3 or | S3 (lovastatin) | CETP1 (torcetrapib) |
| M4 or M5 or M6 | | |
| M1 or M2 or M3 or | S4 (pravastatin) | CETP1 (torcetrapib) |
| M4 or M5 or M6 | | |
| M1 or M2 or M3 or | S5 (rosuvastatin) | CETP1 (torcetrapib) |
| M4 or M5 or M6 | | |
| M1 or M2 or M3 or | S6 (pitavastatin) | CETP1 (torcetrapib) |
| M4 or M5 or M6 | | |
| M1 or M2 or M3 or | S7 (tenivastatin) | CETP1 (torcetrapib) |
| M4 or M5 or M6 | | |
| M1 or M2 or M3 or | S8 (simvastanin) | CETP1 (torcetrapib) |
| M4 or M5 or M6 | | |
| M1 or M2 or M3 or | S9 (rivastatin) | CETP1 (torcetrapib) |
| M4 or M5 or M6 | | |
| M1 or M2 or M3 or | S10 (mevastatin) | CETP1 (torcetrapib) |
| M4 or M5 or M6 | | |
| M1 or M2 or M3 or | S11 (cerivastatin) | CETP1 (torcetrapib) |
| M4 or M5 or M6 | | |
| M1 or M2 or M3 or | S1 (atorvastatin) | CETP2 (JTT-705) |
| M4 or M5 or M6 | | |
| M1 or M2 or M3 or | S2 (fluvastatin) | CETP2 (JTT-705) |
| M4 or M5 or M6 | | |
| M1 or M2 or M3 or | S3 (lovastatin) | CETP2 (JTT-705) |
| M4 or M5 or M6 | | |
| M1 or M2 or M3 or | S4 (pravastatin) | CETP2 (JTT-705) |
| M4 or M5 or M6 | | |
| M1 or M2 or M3 or | S5 (rosuvastatin) | CETP2 (JTT-705) |
| M4 or M5 or M6 | | |
| M1 or M2 or M3 or | S6 (pitavastatin) | CETP2 (JTT-705) |
| M4 or M5 or M6 | | |
| M1 or M2 or M3 or | S7 (tenivastatin) | CETP2 (JTT-705) |
| M4 or M5 or M6 | | |
| M1 or M2 or M3 or | S8 (simvastanin) | CETP2 (JTT-705) |
| M4 or M5 or M6 | | |
| M1 or M2 or M3 or | S9 (rivastatin) | CETP2 (JTT-705) |
| M4 or M5 or M6 | | |
| M1 or M2 or M3 or | S10 (mevastatin) | CETP2 (JTT-705) |
| M4 or M5 or M6 | | |
| M1 or M2 or M3 or | S11 (cerivastatin) | CETP2 (JTT-705) |
| M4 or M5 or M6 | | |

It is understood that, as shown in TABLE 11, the MTP inhibitor present in each triple combination can be an MTP inhibitor denoted as M1 or M2 or M3 or M4 or M5 or M6.

Exemplary combinations of an MTP inhibitor, a fibrate and a squalene synthetase inhibitors are denoted in TABLE 12.

TABLE 12

| | | SQUALENE |
|---|---|---|
| | | SYNTHETASE |
| MTP INHIBITOR | FIBRATE | INHIBITOR |
| M1 or M2 or M3 or | F1 (fenofibrate) | SQ1 (TAK-475) |
| M4 or M5 or M6 | | |
| M1 or M2 or M3 or | F2 (bezafibrate) | SQ1 (TAK-475) |
| M4 or M5 or M6 | | |
| M1 or M2 or M3 or | F3 (ciprofibrate) | SQ1 (TAK-475) |
| M4 or M5 or M6 | | |
| M1 or M2 or M3 or | F4 (clofibrate) | SQ1 (TAK-475) |
| M4 or M5 or M6 | | |
| M1 or M2 or M3 or | F5 (gemfibrozil) | SQ1 (TAK-475) |
| M4 or M5 or M6 | | |
| M1 or M2 or M3 or | F1 (fenofibrate) | SQ2 (ER-27856) |
| M4 or M5 or M6 | | |
| M1 or M2 or M3 or | F2 (bezafibrate) | SQ2 (ER-27856) |
| M4 or M5 or M6 | | |
| M1 or M2 or M3 or | F3 (ciprofibrate) | SQ2 (ER-27856) |
| M4 or M5 or M6 | | |
| M1 or M2 or M3 or | F4 (clofibrate) | SQ2 (ER-27856) |
| M4 or M5 or M6 | | |
| M1 or M2 or M3 or | F5 (gemfibrozil) | SQ2 (ER-27856) |
| M4 or M5 or M6 | | |
| M1 or M2 or M3 or | F1 (fenofibrate) | SQ3 (RPR-107393) |
| M4 or M5 or M6 | | |
| M1 or M2 or M3 or | F2 (bezafibrate) | SQ3 (RPR-107393) |
| M4 or M5 or M6 | | |
| M1 or M2 or M3 or | F3 (ciprofibrate) | SQ3 (RPR-107393) |
| M4 or M5 or M6 | | |
| M1 or M2 or M3 or | F4 (clofibrate) | SQ3 (RPR-107393) |
| M4 or M5 or M6 | | |
| M1 or M2 or M3 or | F5 (gemfibrozil) | SQ3 (RPR-107393) |
| M4 or M5 or M6 | | |

It is understood that, as shown in TABLE 12, the MTP inhibitor present in each triple combination can be an MTP inhibitor denoted as M1 or M2 or M3 or M4 or M5 or M6.

Exemplary combinations of an MTP inhibitor, a fibrate and an ACAT inhibitor are denoted in TABLE 13.

TABLE 13

| MTP INHIBITOR | FIBRATE | ACAT INHIBITOR |
|---|---|---|
| M1 or M2 or M3 or | F1 (fenofibrate) | ACAT1 (avasimibe) |
| M4 or M5 or M6 | | |
| M1 or M2 or M3 or | F2 (bezafibrate) | ACAT1 (avasimibe) |
| M4 or M5 or M6 | | |
| M1 or M2 or M3 or | F3 (ciprofibrate) | ACAT1 (avasimibe) |
| M4 or M5 or M6 | | |
| M1 or M2 or M3 or | F4 (clofibrate) | ACAT1 (avasimibe) |
| M4 or M5 or M6 | | |
| M1 or M2 or M3 or | F5 (gemfibrozil) | ACAT1 (avasimibe) |
| M4 or M5 or M6 | | |
| M1 or M2 or M3 or | F1 (fenofibrate) | ACAT2 (pactimibe) |
| M4 or M5 or M6 | | |
| M1 or M2 or M3 or | F2 (bezafibrate) | ACAT2 (pactimibe) |
| M4 or M5 or M6 | | |
| M1 or M2 or M3 or | F3 (ciprofibrate) | ACAT2 (pactimibe) |
| M4 or M5 or M6 | | |
| M1 or M2 or M3 or | F4 (clofibrate) | ACAT2 (pactimibe) |
| M4 or M5 or M6 | | |
| M1 or M2 or M3 or | F5 (gemfibrozil) | ACAT2 (pactimibe) |
| M4 or M5 or M6 | | |
| M1 or M2 or M3 or | F1 (fenofibrate) | ACAT3 (HL-004) |
| M4 or M5 or M6 | | |
| M1 or M2 or M3 or | F2 (bezafibrate) | ACAT3 (HL-004) |
| M4 or M5 or M6 | | |
| M1 or M2 or M3 or | F3 (ciprofibrate) | ACAT3 (HL-004) |
| M4 or M5 or M6 | | |
| M1 or M2 or M3 or | F4 (clofibrate) | ACAT3 (HL-004) |
| M4 or M5 or M6 | | |
| M1 or M2 or M3 or | F5 (gemfibrozil) | ACAT3 (HL-004) |
| M4 or M5 or M6 | | |

It is understood that, as shown in TABLE 13, the MTP inhibitor present in each triple combination can be an MTP inhibitor denoted as M1 or M2 or M3 or M4 or M5 or M6.

Exemplary combinations of an MTP inhibitor, a fibrate and a CETP inhibitor are denoted in TABLE 14.

TABLE 14

| MTP INHIBITOR | FIBRATE | CETP INHIBITORS |
|---|---|---|
| M1 or M2 or M3 or | F1 (fenofibrate) | CETP1 (torcetrapib) |
| M4 or M5 or M6 | | |
| M1 or M2 or M3 or | F2 (bezafibrate) | CETP1 (torcetrapib) |
| M4 or M5 or M6 | | |
| M1 or M2 or M3 or | F3 (ciprofibrate) | CETP1 (torcetrapib) |
| M4 or M5 or M6 | | |
| M1 or M2 or M3 or | F4 (clofibrate) | CETP1 (torcetrapib) |
| M4 or M5 or M6 | | |
| M1 or M2 or M3 or | F5 (gemfibrozil) | CETP1 (torcetrapib) |
| M4 or M5 or M6 | | |
| M1 or M2 or M3 or | F1 (fenofibrate) | CETP2 (JTT-705) |
| M4 or M5 or M6 | | |
| M1 or M2 or M3 or | F2 (bezafibrate) | CETP2 (JTT-705) |
| M4 or M5 or M6 | | |
| M1 or M2 or M3 or | F3 (ciprofibrate) | CETP2 (JTT-705) |
| M4 or M5 or M6 | | |
| M1 or M2 or M3 or | F4 (clofibrate) | CETP2 (JTT-705) |
| M4 or M5 or M6 | | |
| M1 or M2 or M3 or | F5 (gemfibrozil) | CETP2 (JTT-705) |
| M4 or M5 or M6 | | |

It is understood that, as shown in TABLE 14, the MTP inhibitor present in each triple combination can be an MTP inhibitor denoted as M1 or M2 or M3 or M4 or M5 or M6.

Exemplary combinations of an MTP inhibitor, a niacin and a squalene synthetase inhibitor are denoted in TABLE 15.

TABLE 15

| | | SQUALENE |
|---|---|---|
| | | SYNTHETASE |
| MTP INHIBITOR | NIACIN | INHIBITOR |
| M1 or M2 or M3 or | N1 (niacin) | SQ1 (TAK-475) |
| M4 or M5 or M6 | | |
| M1 or M2 or M3 or | N1 (niacin) | SQ2 (ER-27856) |
| M4 or M5 or M6 | | |
| M1 or M2 or M3 or | N1 (niacin) | SQ3 (RPR-107393) |
| M4 or M5 or M6 | | |

It is understood that, as shown in TABLE 15, the MTP inhibitor present in each triple combination can be an MTP inhibitor denoted as M1 or M2 or M3 or M4 or M5 or M6.

Exemplary combinations of an MTP inhibitor, a niacin and an ACAT inhibitor are denoted in TABLE 16.

TABLE 16

| MTP INHIBITOR | NIACIN | ACAT INHIBITOR |
|---|---|---|
| M1 or M2 or M3 or | N1 (niacin) | ACAT1 (avasimibe) |
| M4 or M5 or M6 | | |
| M1 or M2 or M3 or | N1 (niacin) | ACAT2 (pactimibe) |
| M4 or M5 or M6 | | |
| M1 or M2 or M3 or | N1 (niacin) | ACAT3 (HL-004) |
| M4 or M5 or M6 | | |

It is understood that, as shown in TABLE 16, the MTP inhibitor present in each triple combination can be an MTP inhibitor denoted as M1 or M2 or M3 or M4 or M5 or M6.

Exemplary combinations of an MTP inhibitor, a niacin and CETP inhibitors are denoted in TABLE 17.

TABLE 17

| MTP INHIBITOR | NIACIN | CETP INHIBITOR |
|---|---|---|
| M1 or M2 or M3 or | N1 (niacin) | CETP1 (torcetrapib) |
| M4 or M5 or M6 | | |
| M1 or M2 or M3 or | N1 (niacin) | CETP2 (JTT-705) |
| M4 or M5 or M6 | | |

It is understood that, as shown in TABLE 17, the MTP inhibitor present in each triple combination can be an MTP inhibitor denoted as M1 or M2 or M3 or M4 or M5 or M6.

Formulations (with one, two, three or more active ingredients), such as those set forth herein, may prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes but is not limited to diluents, binders, lubricants, disintegrators, fillers, and coating compositions.

Furthermore, it is contemplated that the compositions can be formulated as extended release formulations, for example, prepared as diffusion or osmotic systems, for example, as described in “Remington—The science and practice of pharmacy” (20th ed., Lippincott Williams & Wilkins, Baltimore, Md., 2000). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and carbopol 934, polyethylene oxides. Fatty compounds include, but are not limited to, various waxes such as carnauba wax and glyceryl tristearate

Typical dosage ranges for each of the components of the combinations are set forth below. Dosage ranges for the MTP inhibitor include, for example, 0.5 to 160 mg/day, more particularly 0.01 to 1, 2.5, 5, 7.5, 10, 15, 20 30, 40, or 80 mg. For example, BMS-201038 may be administered with a dose of about 2.5 mg/day to about 10 mg/day, or to about 25 mg/day or more, for example 2.5 mg/day, 5 mg/day or 10 mg/day. Implitapide may be administered, for example dosages including 20-60 mg/day, 20-50 mg/day, or 20-30 mg/day, for example, 20 mg/day, 35 mg/day, or 45 mg/day.

Dosage ranges for CAI include, for example, 1 to 50 mg/day, more particularly 0.01 to 1, 2.5, 5, 7.5, 10, 15, 20, or 25 mg/day. Dosage ranges for the HMG-CoA reductase inhibitor include, for example, 1 mg/day to about 20, 25, 30, 40, 50, 60, 70 or 80 mg/day. Dosage ranges for the fibrates include, for example, 10 mg/day to 750 mg/day, 25 mg/day to 500 mg/day and 25 mg/day to 200, 225, 250, 300, 325, 350 or 400 mg/day. Dosages ranges for the squalene synthetase inhibitors include, for example, 10 to 200 mg/day, more particularly 10 to 50 mg/day. Dosages ranges for niacin include, for example, 100 mg-10 g/day, more particularly 250 mg to 7 g/day. Dosage ranges for other cholesterol lowering drugs are within the dosage ranges where the particular active ingredients are safe and/or effective in humans. In addition, it is contemplated that the MTP inhibitors and/or the other cholesterol lowering agents may be administered at escalating doses.

In some embodiments, the active ingredient, or cocktail of active ingredients, are administered daily. The duration, however, may range from weeks, to months, to years, depending upon the circumstances, for example, during the treatment of chronic disorder. For example, the active ingredients may be administered for 1 day to 1 week, 1 day to 2 weeks, 1 day to 3 weeks, 1 day to 4 weeks, 1 days to 5 weeks, 1 day to 6 weeks, 1 day to 7 weeks, 1 day to 2 months, 3 months, 4 months, 5 months, 6 months, 7 months, 8 months, 9 months, 10 months, 11 months, or 12 months, and for 2, 3, 4, 5, 6, or more years.

### (3) Modes and Administration

It is contemplated that the active ingredients may be administered at the same time or sequentially.

When the active ingredients are administered at the same time, they may be administered in the same formulation or in different formulations. When administered in the same formulation, the active ingredients will be administered by the same route, for example, by a parenteral or a non-parenteral route. When administered in different formulations, one formulation may be administered by one route, for example, by a parenteral route whereas the other active ingredient may be administered by one or more non-parenteral routes. Alternatively, all the formulations may be administered by the same route.

When the active ingredients are administered sequentially, they can be administered by the same or different routes.

### (4) Methods of Use

The present invention provides methods and compositions for treating diseases or disorders associated with elevated levels of cholesterol and triglycerides in blood. For example, the compositions and methods can be used for preventing, inhibiting or treating atherosclerosis, pancreatitis, obesity, hyperlipidemia (including, for example, hypercholesterolemia and hypertriglyceridemias).

The methods disclosed herein may reduce or lower the concentration of serum cholesterol. It is understood that total serum cholesterol can be provided by very low density lipoproteins (VLDL), intermediate density lipoproteins (IDL), LDL and chylomicrons. Accordingly, it is contemplated that the combination therapies may reduce total blood cholesterol, or cholesterol provided by or associated with VLDL, IDL, LDL and chylomicrons. In addition, the methods disclosed herein may reduce or lower the concentration of serum triglycerides. It is understood that the serum triglycerides can be provided by VLDL and chylomicrons, and to a lesser extent by IDL and LDL. Accordingly, it is contemplated that the combination therapies may reduce triglycerides provided by or associated with VLDL, IDL, LDL and chylomicrons.

In certain embodiments, the compositions described herein may provide therapeutic benefit in preventing, inhibiting or treating one of the foregoing disorders while at the same time minimizing at least one of nausea, vomiting, steatorrhea, abdominal cramping, distention, elevated liver function tests, fatty liver; hepatic fat build up, polyneuropathy, peripheral neuropathy, rhabdomyolysis, arthralgia, myalgia, chest pain, rhinitis, dizziness, arthritis, peripheral edema, gastroenteritis, liver function tests abnormal, colitis, rectal hemorrhage, esophagitis, eructation, stomatitis, biliary pain, cheilitis, duodenal ulcer, dysphagia, enteritis, melena, gum hemorrhage, stomach ulcer, tenesmus, ulcerative stomatitis, hepatitis, pancreatitis, cholestatic jaundice, paresthesia, amnesia, libido decreased, emotional lability, incoordination, torticollis, facial paralysis, hyperkinesia, depression, hypesthesia, hypertonia, leg cramps, bursitis, tenosynovitis, myasthenia, tendinous contracture, myositis, hyperglycemia, creatine phosphokinase increased, gout, weight gain, hypoglycemia, anaphylaxis, angioneurotic edema, and bullous rashes (including erythema multiforme, Stevens-Johnson syndrome, and toxic epidermal necrolysis). In some embodiments the minimization of the side effect is determined by assessing the grade, severity, extent, or duration by subject questionnaire.

It is also contemplated that the compositions described herein can be useful in slowing or stopping the development and/or stabilizing plaques, for example, arterial plaques, on the wall of a blood vessel. In addition, it is contemplated that under certain circumstances, the compositions described herein, will effectively induce plaque regression. Under certain circumstances, the level of plaque regression may result in 20%, 30%, 40%, 50%, 60%, 70%, 80%, 90%, 95%, and 99% of the original plaque load disappearing after the start of therapy. The extent arterial plaques, and the reduction thereof, can be measured using a variety of conventional non-invasive techniques known in the art, including, for example, magnetic resonance imaging, computerized tomography, and nuclear scintigraphy.

The methods and compositions described herein are particularly useful for treating patients, for example, LDL reduction-resistant patients, unable to achieve the cholesterol and/or LDL cholesterol goals desired by their physician and/or outlined by guidelines, for example, the guidelines provided by NCEP. This inability may be due to an inability to tolerate an MTP inhibitor (e.g., BMS-201038) and/or a CAI (e.g., ezetimibe), or the inability of existing agents to provide sufficient cholesterol lowering to achieve these goals (for example, the active ingredient works as it should but too much active ingredient is required achieve the desired end point). The methods and compositions described herein are especially useful for higher risk patients, for example, coronary heart disease patients or patients with a similar risk of a coronary event. Such patients may have a 10 year risk of a coronary event of greater than 20%.

For example, the disclosed methods can include LDL reduction-resistant patients, for example, coronary heart disease or coronary heart disease risk equivalent patients with severe hypercholesterolemia of any etiology unable to come within 25%, more preferably 15%, of their NCEP LDL cholesterol goal on maximal tolerated oral therapy, as determined by their prescribing physician based upon established guidelines.

In another embodiment, the methods and composition described herein may be used for the treatment of severe hypercholesterolemia of any etiology unable to meet the goal of 75 mg/dL of NCEP LDL cholesterol goal on maximal tolerated oral therapy.

The combination of a MTPI, CAI and a HMG-CoA reductase inhibitor may be useful in treating a CHD or CHD risk equivalent (RE) patient unable to meet the NCEP goal of 70 mg/dL on statin monotherapy or statin combination therapy with a CAI. For example, a study presented at the American College of Cardiology meeting in Orlando in March 2005 by Christie Ballantyne M.D, demonstrated that Lipitor at a dose of 40 mg only resulted in 23% of the patients studied achieving goal attainment, i.e., <70 mg/dL, and the combination of a statin plus CAI, Vytorin 10/40 (10 mg of ezetimibe, a CAI, and 40 mg of simvastatin, a statin, achieved LDL <70 mg/dL in only 57% of patients. Therefore, a significant percentage of patients require more than statin monotherapy or statin in combination with CAI to achieve the NCEP goal. The combinations described herein should allow goal attainment in potentially greater than 90% of high risk patients, which would be a marked improvement over existing therapies.

The methods disclosed herein may include patients with severe hypertriglyceridemia unable to reduce total triglyceride levels to <1000 mg/dL or <500 mg/dL on maximal tolerated therapy. Under certain circumstances, the triple combination therapy described herein will permit individuals to reach a goal of having less than 70 mg/dL of LDL, 60 mg/dL of LDL, 50 mg/dL of LDL, 40 mg/dL of LDL, 30 mg/dL of LDL, 20 mg/dL of LDL, or 10 mg/dL of LDL in their blood during fasting.

### EQUIVALENTS

It is understood that the disclosed invention is not limited to the particular methodology, protocols, and reagents described as these may vary. It is also to be understood that the terminology used herein is for the purpose of describing particular embodiments only, and is not intended to limit the scope of the present invention which will be limited only by the appended claims.

### INCORPORATION BY REFERENCE

The entire disclosure of each of the patent documents and scientific articles referred to herein is incorporated by reference for all purposes.

1. A pharmaceutical composition comprising (i) a microsomal triglyceride transfer protein inhibitor (“MTPI”), (ii) cholesterol absorption inhibitor (CAI), and (iii) at least one cholesterol lowering drug selected from the group consisting of a HMG-CoA reductase inhibitor, a bile acid sequestrant, a fibric acid derivative, niacin, and a squalene sythetase inhibitor.

2. A pharmaceutical composition comprising (i) an MTPI, (ii) a HMG-CoA reductase inhibitor, and (iii) at least one cholesterol lowering drug selected from the group consisting of a bile acid sequestrant, a fibric acid derivative, niacin, and a squalene sythetase inhibitor.

3. The composition of claim 1, wherein the MTPI is BMS-201038, implitapide, JTT-130 and CP-346086.

4. The composition of claim 1, wherein the MTPI is BMS-201038.

5. The composition of claim 1, wherein the MTPI is implitapide.

6. The composition of claim 3, wherein the HMG-CoA reductase inhibitor is selected from the group consisting of mevastatin, lovastatin, pravastatin, simvastatin, fluvastatin, cerivastatin, atorvastatin, tenivastatin, rosuvastatin, pitavastatin and combinations thereof.

7. The composition of claim 6, wherein the HMG-CoA reductase inhibitor is simvastatin or atorvastatin.

8. The composition of claim 1, wherein the CAI is selected from the group consisting of ezetimibe or a derivative thereof, MD-0727, and FM-VP4.

9. The composition of claim 8, wherein the CAI is ezetimibe.

10. The composition of claim 1, wherein the bile acid sequestrant is selected from the group consisting of cholestyramine, colesevelam and colestipol.

11. The composition of claim 1, wherein the fibric acid derivative is selected from the group consisting of fenofibrate and gemfibrozil.

12. A pharmaceutical composition comprising (i) an MTPI, (ii) a CAI, and (iii) a HMG-CoA reductase inhibitor.

13. The composition of claim 12 further comprising a cholesterol lowering drug selected from the group consisting of a bile acid sequestrant, a fibric acid derivative, and niacin.

14. The composition of claim 12, further comprising one or more pharmaceutically acceptable carriers.

15. The composition of claim 14, wherein the one or more pharmaceutically acceptable carriers is selected from the group consisting of diluents, binders, lubricants, solubilizing agents, stabilizing agents, disintegrators, fillers, surfactants, and coating formulations.

16. The composition of claim 14, wherein the formulation is intended for oral administration.

17. A method of lowering the concentration of serum cholesterol in a mammal, the method comprising administering to the mammal an effective amount of the composition of claim 1.

18. A method of lowering the concentration of serum triglycerides in a mammal, the method comprising administering to the mammal an effective amount of the composition of claim 1.

19. The method of treating hyperlipidemia in a mammal, the method comprising administering to the mammal an effective amount of the composition of claim 1.

20. A method of lowering the concentration of serum cholesterol and/or serum triglycerides in a mammal, the method comprising administering three active ingredients comprising (i) and MTP inhibitor, (ii) a CAI, and (iii) at least one cholesterol lowering drug selected from the group consisting of a HGM-CoA reductase inhibitor, a bile acid sequestrant, a fibric acid derivative, niacin, and squalene sythetase inhibitor.

21. A method of lowering the concentration of serum cholesterol and/or serum triglycerides in a mammal, the method comprising administering three active ingredients comprising (i) and MTP inhibitor, (ii) a HGM-CoA reductase inhibitor, and (iii) at least one cholesterol lowering drug selected from the group consisting of a bile acid sequestrant, a fibric acid derivative, niacin, and squalene sythetase inhibitor.

22. A method of include the regression of plaques on the wall of a blood vessel in a mammal, the method comprising administering three active ingredients comprising (i) and MTP inhibitor, (ii) a CAI, and (iii) at least one cholesterol lowering drug selected from the group consisting of a HGM-CoA reductase inhibitor, a bile acid sequestrant, a fibric acid derivative, niacin, and squalene sythetase inhibitor so as to induce regression of the plaques.

23. A method of include the regression of plaques on the wall of a blood vessel in a mammal, the method comprising administering three active ingredients comprising (i) and MTP inhibitor, (ii) a HGM-CoA reductase inhibitor, and (iii) at least one cholesterol lowering drug selected from the group consisting of a bile acid sequestrant, a fibric acid derivative, niacin, and squalene sythetase inhibitor so as to induce regression of the plaques.

24. The method claim 20, wherein at least two of the active ingredients are administered at the same time.

25. The method of claim 20, wherein three of the active ingredients are administered at the same time.

26. The method of claim 20, wherein all three active ingredients are administered sequentially.

27. The method of claim 20, wherein the active ingredients, when administered at the same time, are administered in the same formulation.

28. The method of claim 20, wherein the active ingredients, when administered at the same time, are administered in different formulations.
